(19) United States
(12) Patent Application Publication
(10) Pub. No.: US 2018/0236123 A1
(43) Pub. Date: Aug. 23, 2018

(54) Hemostatic Composition and Hemostatic Device (Variants)

(71) Applicants: Petro Andriyovych Manoryk, Kyiv (UA); Vadym Borysovych Mazevych, Brovary (UA); Igor Sergiyovych Tsurupa, Obukhiv (UA); Ihor Volodymyrovych Gaiovych, Kyiv (UA); Svitlana Oleksandrivna Sotnik, Crimea (UA); Volodimir Mykolayovych Granich, Kyiv (UA); laroslav Viktorovych Kyshenia, Kyiv (UA)

(72) Inventors: Petro Andriyovych Manoryk, Kyiv (UA); Vadym Borysovych Mazevych, Brovary (UA); Igor Sergiyovych Tsurupa, Obukhiv (UA); Ihor Volodymyrovych Gaiovych, Kyiv (UA); Svitlana Oleksandrivna Sotnik, Crimea (UA); Volodimir Mykolayovych Granich, Kyiv (UA); laroslav Viktorovych Kyshenia, Kyiv (UA)

(21) Appl. No.: 15/551,437

(22) PCT Filed: Feb. 15, 2016
(86) PCT No.: PCT/UA2016/000019

§ 371 Date: Aug. 16, 2017

(30) Foreign Application Priority Data
Feb. 16, 2015 (UA) A 2015 01285
Feb. 16, 2015 (UA) U 2015 01288

(51) Int. Cl. A61L 15/44; A61L 15/54; A61L 26/00

(52) U.S. Cl. CPC A61L 15/44; A61L 15/54; A61L 2400/04; A61L 2300/418; A61L 26/0066

(57) ABSTRACT

The hemostatic composition, comprising water-retaining, binder dust suppression, inorganic and organic hemostatic agents, and hemostatic device comprising the composition of hemostatic agents and a container that keeps hemostatic composition.

### BACKGROUND

The invention relates to medical devices.

Detailed, the invention relates to medical first aid kits in body injuries, accompanied by bleeding, including heavy bleeding.

More detailed, the invention relates to medical first aid kits in body injuries received particularly heavy injuries as well as injuries, including wounds received during the war military operations.

The composition and device are designed to provide first aid for said injuries, but not limited to, accompanied by bleeding, to stop such bleeding. Composition and device are designed to instantly stop bleeding and to create conditions for the safe and most rapid transportation of wounded/injured, that is ensured by specific hemostatic properties of composition and device.

The invention relates to a composition of hemostatic agents and hemostatic device, consisting of said composition and container, and provide the most efficient use of them at the stage of self-aid, mutual aid and first and unskilled premedical and qualified medical aid to stop the bleeding, heavy bleeding, including shrapnel and gunshot wounds, received mainly on the battlefield, which could to equip military individual first aid kit of various types and as well as in surgery and traumatology.

Such hemostatic compositions and hemostatic devices intended for:

quick stop massive bleeding, including caused by shrapnel and gunshot wounds, mainly received on the battlefield on the stage of self-aid, mutual aid and first unskilled premedical and professional medical aid, which can equip military individual first aid kit of various levels;

for surgery, traumatology and disaster medicine.

The composition of hemostatic agents due to its qualitative and quantitative composition should provide the following:

Due to presence of several hemostatic agents acting separately to its specific coagulation factor and, in combination with other agent, mutually enhance the action of other agent providing synergistic effect of composition,

the composition of hemostatic agents with its properties should be suitable to be combined with a carrier (to be incorporated into the carrier) without losing its hemostatic properties and due to their physicochemical and physical properties provided (not prevented) process of manufacturing hemostatic devices.

In their turn hemostatic agents through a combination of composition, which has a certain chemical, physicochemical and physical properties, and the container as a gauze substrate and/or vessel and/or water permeable film, that has certain physical, mechanical and chemical, physicochemical and physical properties into one and using appropriate compounds, substances and materials the composition and carrier made, suitable design of hemostatic device, including the use of additional elements of design, including in particular, introducers, applicators, containers, adhesive materials, radiographic materials, packaging, etc. should provide:

contact of hemostatic agents of the composition incorporated into the container with blood to accelerate its clotting after contact of hemostatic devices with blood;

tightness of packaging and sterility of hemostatic devices during storage, transport and in some cases its use;

keep properties of hemostatic device in harsh climate conditions (from −40° to ++50° C., under humidity over 95%) during storage, transportation and use,

radiographic properties of the device for the identification of a device or its fragments in the wound;

increasing the efficiency of hemostatic composition of hemostatic agents, part of the hemostatic device, as compared with the composition itself;

convenience and reliability of plugging, convenience of removing device from the wound and reduce the time required for plugging the wound;

synergic acceleration of blood clotting time due the fact that the components of hemostatic device (hemostatic agents of the hemostatic composition and container) not only each by itself it accelerates clotting, but also enhances the action of each other due to the impact at the same time on various blood clotting factors.

Hemostatic agents and devices designed for use in combat conditions to stop the massive bleeding should be convenient to use, be compact for transportation and storage before use, to provide the most rapid and reliable plugging of the wound.

### Glossary

These terms when used herein have the following meanings.

The term “Hemostatic agent” as used herein, means a material, matter, chemical compound, a mineral of natural origin and/or the product of their chemical and/or biological and/or chemical modification, and/or of synthetic origin, which contacting with blood initiates and/or accelerates its clotting by any mechanism.

The term “Hemostatic composition” as used herein, means a matter simultaneously consisting of the four said hemostatic, which are characterized as water-retaining, binder dust suppression, inorganic and organic hemostatic, and which are in defined ratio and their total content in the said hemostatic composition is less than 100% or 100%, and where the said hemostatic composition is a liquid or a solid, and is characterized by that it is in form of solution or suspension or foam or gel or paste or powder, which contacting with blood initiates and/or accelerates its clotting by any mechanism.

The term “Hemostatic device” as used herein, means in the sense of the present invention is a material that is made and consists of container (substrate) and the composition of hemostatic agents in any rational way combined, enabling its use to control bleeding.

The term “Two-dimensional container”, “two-dimensional substrate”, “two-dimensional device” as used herein, means any container (substrate) and device, which are being two-dimensional geometrical structure (shape) preferably, but not exclusively, flat, whose dimensions in length and width far exceed height, for example, having Length or Width to Height ratio at least 10 times, including those which for compact packaging (such as napkins, bandages) are folded or rolled in a three-dimensional shapes, such as corrugations, rolls or for accessibility (e.g., cord, helix) or as examples of two-dimensional, flat structures can be gauzes, bandages, napkins and more.

The term “Additional agents” as used herein, means any agents that provide additional functional properties of the said hemostatic composition or the said hemostatic device. Examples of such agents may include providing radiographic properties of the composition and/or device, for example, barium salts, pharmaceutically-active agents which are antibiotics, antifungal agents, antimicrobial agents, anti-inflammatory agents, analgesics, analgesics and anesthetics of local action, antihistamines, compounds and/or matters containing ions of copper, zinc, silver, gold and combinations thereof and additional agents which are own hemostatics, e.g. thrombin, tranexamic acid, ε-aminocaproic acid, bioactive glass, biological hemostatics and combinations thereof, and also polyvinyl alcohol, glycerol, silicone, carboxymethyl cellulose, gelatinized starch, polyacrylic acid and its salts of iron, calcium, barium.

The term “Three-dimensional container” and “Three-dimensional substrate” as used herein, means any container and substrate, which are mainly have three-dimensional geometric shape, but not exclusively, cylinder, sphere, ellipsoid, box and so on. in the form of discs, balls, pockets, sacs, pads, which are made at least of one piece of two-dimensional substrate by fixing at least two opposite edges of the two-dimensional substrate in any rational way and sprays, syringes, tubes, containers and other vessels including sealed for storage of liquid, semi-liquid, gel-like and paste-like and foam-like and other suitable forms of the hemostatic composition.

The term “Three-dimensional hemostatic device” as used herein, means any device having three-dimensional geometric shape preferably, but not exclusively, cylinder, sphere, ellipsoid, a box in the form of discs, balls, pockets, sacs, pads, tubes made at least of a fragment of a two-dimensional agent or device, by fixing at least two opposite edges of the two-dimensional devices any rational way and sprays, syringes, tubes, containers and other vessels, including sealed, mainly filled with solid, liquid, semi-liquid, gel-like and paste-like and foam-like spumy and other suitable forms of hemostatic composition, which can also include additional agents having three-dimensional geometric shape preferably, but not exclusively, cylinder, sphere, ellipsoid, a box.

The term “Composition of hemostatic” or “Hemostatic composition” as used herein, means solution, suspension, paste, gel, foam, dry (or semi dry) matter, which is obtained by any rational way of hemostatic agents.

The terms “Container” and “Substrate” as used herein, are used interchangeably and mean a facility that can be soaked, filled, loaded or otherwise combined with hemostatic composition so that it makes possible to use hemostatic agents compositions combined with the container (substrate) as hemostatic device.

The term “Container” as used herein, is a two-dimensional, mainly flat structure, whose dimensions in length and width far exceed in height, for example, having Length or Width to Height ratio as at least 10 times, or three-dimensional structure, preferably so voluminous structure of different types. Examples of two-dimensional, flat structures can be gauzes, bandages, napkins etc. Examples of three-dimensional structures can be various structures such as tampons, vessels, devices for introduction into the cavities and hollows, sealed vessels for storing liquid and semi-liquid, gel-like and paste-like and other suitable forms of hemostatic agents composition.

The term “Slot-dye process” as used herein, is a process used for the application of a viscous hemostatic composition or individual hemostatic agents of said composition and/or combinations thereof, on the “two-dimensional” substrate preferably in the form of a tape, using a special slit extrusion head by squeezing through it the hemostatic composition or components that constitute it, on the “two-dimensional” substrate that moves with the optimum at any given time speed.

### BACKGROUND

There are currently agents affecting blood clotting system and have hemostatic effect, divided into different groups and different on mechanism of action (M [symbol] [symbol] [symbol] [symbol] [symbol] . ( [symbol] [symbol] [symbol] ). [symbol] , [symbol] [symbol] 1996, c.78-103): Antihemorrhagic and hemostatic agents, including: heparin antagonists, such as protamine sulfate, which is the medicine preparation of protein origin, which is a specific antagonist of heparin, an anticoagulant of blood which is directly applicable, and is used mainly as necessary to neutralize the effect of excess exogenous heparin; inhibitors of fibrinolysis, such as, ε-aminocaproic acid and ambene which are inhibitors of fibrinolysis, that is the process that primarily causes destruction of fibrinous thread. ( [symbol] [symbol] [symbol] ) [symbol] , [symbol] [symbol] 1996, c.96-99). [symbol] , [symbol] [symbol] 1996, c.78-103): antihemorrhagic and hemostatic agents, including: heparin antagonists, such as protamine sulfate is the medicine preparation of protein origin is a specific antagonist of heparin is directly applicable anticoagulant of blood and mainly used if it required to neutralize the effect of excess exogenous heparin; inhibitors of fibrinolysis, such as, ε-aminocaproic acid and Ambene are inhibitors of fibrinolysis, the process primarily causing destruction of fibrinous thread. Fibrinogen and thrombin are native components of blood clotting among of the key factors triggering fibrin thread formation and platelet adhesion. ε-Aminocaproic acid is substance blocking plasminogen activators and partially inhibits the action of plasmin and therefore may have specific bleeding control action in bleeding associated with increase of fibrinolysis, it also inhibits kinins—biogenic polypeptides generated in the body of α-globulins under kallikrein. Preparation used to bleeding control during surgical intervention and various pathological conditions in which there is an increased fibrinolytic activity of blood and tissue; Ambene (para(aminomethyl) benzoic acid) inhibits fibrinolysis by competitive inhibition of plasminogen activating enzyme and inhibition of plasmin formation. Preparation used to bleeding control during surgical intervention and various pathological conditions in which increased fibrinolytic activity of blood and tissues, as well as hemorrhagic diathesis protocytopenic origin; vasoprotector such as calcium dobezylat, belonging to a group of vasoprotectors and has the effect of proagregation action increasing platelets activity as hemostatic agents; clot-forming agents, such as decylat (Trombovar, Varicol) (2-methyl-7-etylundectsyl-4-sulfate), which is sclerosing preparation having additional surface-active properties, if administered intravenously causes thrombosis with subsequent blood clot organization and its fusion with of vessel wall, and is designed for to sclerosing treatment of varicose veins of the lower extremities.

There are examples of use of substances derived from various animal and human tissues, as hemostatic agents ( [symbol] [symbol] , [symbol] [symbol] [symbol] , ( [symbol] [symbol] [symbol] ). ( [symbol] [symbol] [symbol] ) [symbol] , [symbol] [symbol] 1996, c.96-99). [symbol] , [symbol] [symbol] 1996, c.96-99), including: fibrinogen (from donor's plasma) is natural component of blood under the action of thrombin transforms into fibrin ad carries out end stage of clotting—clot formation. In hypo- and afibrinogenemia, bleeding in traumatology, surgery, oncology, massive bleeding in obstetric and gynecologic practice administered fibrin obtained of fibrinogen of human blood plasma, exhibits hemostatic action, facilitate tissue regeneration and wound healing and its preparations applied locally in surgery, cranial and brain injuries and to fill tissue defects, etc.; thrombin is a natural component of blood clotting system produced in the body by enzymatic activation of prothrombin by thromboplastin and obtained from donors plasma and administered only topically to stop bleeding from small capillaries and small parenchymal organs (in cranial operations, operations on kidneys, liver and other parenchymal organs, bone cavities bleeding, gums, etc., but thrombin not administered in bleeding from large vessels and not allowed into vein and muscle, its administration into blood vessels can cause widespread thrombosis with fatal outcome; collagen and gelatin mainly used in the form of sponges with addition of other functional agents exhibit hemostatic effect and therefore due to ability to resorption can be left in the wound; dried blood plasma, which is obtained from the blood of cattle, reveals hemostatic effect ( [symbol] [symbol] , [symbol] [symbol] [symbol] . ( [symbol] [symbol] [symbol] ) [symbol] , [symbol] [symbol] 1996, c.96-99).

There are examples of vegetable origin to produce hemostatic agents, some of which are used for centuries as hemostatic agents in traditional medicine. There are examples of compounds of plant origin used to produce hemostatic agents, some them used for centuries as hemostatic agents in traditional medicine ( [symbol] [symbol] , [symbol] [symbol] [symbol] . ( [symbol] [symbol] [symbol] ) [symbol] , [symbol] [symbol] 1996, c.99-102), derived in particular from: intoxicating mint (Lagochilus snebrians Bunge) its aerial parts contain lahohilin (tetratomic alcohol), essential oil, tannins, carotene, preparations of which are administered to reduce bleeding during hemorrhagic diathesis, hemorrhoids, nasal and other nasal bleeding; nettle leaves (Folia Urticae), containing ascorbic acid (0.1-0.2%), carotene, vitamin C, tannins, minerals and other substances, including medicines administered in lung, kidney, uterine bleeding and stomach; yarrow herb (Herba Millefolii), which contains the alkaloid achillein, ascorbic acid, carotene, vitamin C, tannins, essential oil, organic acids, resins, preparations of which administered in uterine bleeding and stomach, against the background of inflammation, fibroids, etc.; water pepper herb (Herba Polygoni hydropiperis), which contains quercetin, rutin and other flavonoids, tannins, and where the content (in terms of quercetin) of flavonoids is not less than 0.5%, its preparations are administered to reduce the permeability of blood vessels and improve blood clotting ability; grass Persicaria maculosa (Herba Polygoni persicariae), contains flavonoids, glycosides, ascorbic acid and others, and its preparations administered in bleeding control as a styptic mainly with hemorrhoidal bleeding, the effect of which is associated with moderate hemostatic effect and laxative effect; Viburnum bark (Cortex Viburni opuli), which contains tannins (at least 4%), salts of organic acids and other substances matters and its preparations administered in bleeding control as a styptic mainly in uterine bleeding; arnica (Flores Arnicae), which contain essential oils, tannins, bitter arnicyn, gum, minerals and other substances and preparations of which administered in bleeding control as a styptic in obstetrics and gynecology practice in low regression development of uterus and inflammatory diseases; oak bark containing tannins. Also, these agents are not suitable for use as for massive bleeding control because of their form, storage conditions, poor hemostatic effect, the need to prepare solutions immediately before use and medical supplies are inadequate to the means for a quick stop massive bleeding in large and medium damages (including gunshot and projectile injury), mostly on the battlefield at the point when the self, mutual and unskilled first premedical and medical care.

There hemostatic agents can be divided into 2 types, solid or deposited on the carrier and agents in the form of solutions for external and internal (injectable) application.

Deposited on the carrier can be attributed to two types—chitosan-based and kaolin-based. Various forms of chitosan-based hemostatic agents include products of Celox™ (Medtrade Products Company Ltd, http://www.celoxmedical.com/), Chito-SAM™ (of SAM Medical Products, http://www.sammedical.com/products/chito-sam/) and ChitoGauze™ (of HemCon, http://www.hemcon.com/).

According to the inventors, material Celox™ has some significant drawbacks, such as the presence of granules on the surface of carrier, which can enter the bloodstream through the damaged blood vessels and lead to thrombosis. Also carrier in form of bandage has a considerable thickness and density can complicate tamponade of narrow openings in wounds. Moreover, Celox™ granules have a low hydrophilicity and affinity to blood increases the blood leakage and swelling of material.

The material ChitoGauze™ does not contains granules—thus no possibility of entering to blood flow, but chitosan has a low hydrophilicity.

QuikClot™ belongs to kaolin-based on the carrier—[U.S. Pat. No. 7,604,819 B2, U.S. Pat. No. 8,114,433 B2, U.S. Pat. No. 8,257,732 B2, U.S. Pat. No. 8,383,148 B2, U.S. Pat. No. 8,343,537 B2]. Also known kaolin-based (or zeolites) powder hemostatics—QuikClot™ of first and second generations, Hemostop™ (zeolite with the addition of calcium compounds) (http://www.gemostop.ru/) and HaemoCer™ (modified natural polysaccharide). Powders from dried kaolin or zeolite (QuikClot™ in powder form and bags of powder in waterproof permeable packaging and Hemostop™) resulted in an increase in temperature in the wound environment, resulting in burns and severe painful sensations, as well highly complicated process operating wounds, removal microparticles of active ingredient. Using kaolin applied (stuck) to the carrier eliminates complications of operating wound treatment and previous hydration of the active substance (kaolin) excludes heating the substance in the wound also, on our opinion, hydrophilic material (based only kaolin or zeolite) and its absorbing and adhesive properties are insufficient for fast and successful heavy bleeding control.

Hemostatics also used in form of gel, especially in veterinary medicine, for example Synaero™ Hemostatic Gel (http://www.hemcon.com/Products/Synaero.aspx). Use of gel has several advantages—the density of wounds filling, soft contact with the tissues, ease of surgical cleaning of wounds (opposed to powder), but use of gel with no gel carrier material and its physical action may be not sufficient to stop massive bleeding, such as an arterial.

Liquid hemostatics for external use based on polyacrylic acid salts: Hemolok™ (Ferakryl) (polyacrylate-based iron) and Hemoblok™ (polyacrylate solution of silver) are prescribed to stop minor bleeding, such as capillary, bone or bleeding in dentistry, in principle, unable to stop severe bleeding due to the inability to use liquid tamponade.

Liquid injection hemostatics—Kaprofer™ (iron chloride (Ill), ε-aminocaproic acid, sodium chloride), vitamin K, and other etamsylate and other have biological effects (inhibitors or catalysts of certain processes) for general hemostasis, and may have side effects (such as increasing the chance of thrombosis) so could be prescribed only by qualified physician in hospital environment.

The most common are hemostatic agents (HSA) in dressing form (DF). So, it is known (http://www.znaytovar.ru/s/Klassifikaciya_i_xarakteristika2.html) that hemostatic agents (HSA), namely in dressing form (DF) are ready to use commercial products, and which, depending on the form, belong to group including bandages, packets, napkins, plasters, tampons, sponges, aerosols (foam sprayed, and films that are sprayed) coating for wound; and where the dressing means depending on the form, belong to group including bandages, packets, napkins, plasters, tampons; and where the bandages, which are dressings that are made of cotton viscose gauze in rolls of a certain size; and where the bandages are sized mainly 10 m×6 cm size, 10 m×10 cm, 5 m×10 cm, 5 m×5 cm, 5 m×7 cm, 7 m×10 cm, 7 m×14 cm, 7 m×7 cm and produced both in secondary and individually packed; and where sterile gauze bandages produced 5 m×10 cm size, 5 m×7 cm, 7 m×14 cm individually packed; and where plaster bandages containing gypsum, which after wetting imposed on the injured part of the body for the purpose of fixing, used mostly in traumatology and produced 3 m×10 cm size, 3 m×15 cm, 3 m×20 cm individually packed and which in recent years began to produce PVA with a plasticizer PVA to improve their consumer properties; and

where elastic bandage made of tough cotton yarn, with interwoven rubber threads into base dramatically increases the elasticity of the bandage, and are not sterilized and used for non-rigid shrinkage of soft tissues; and

tubular bandage formed by tube of hydrophilic material, and its elasticity is ensured by knitted weave type, and which is manufactured with a several diameters for use in different parts of the upper and lower extremities; and

where a special kind of tubular bandages are bandages mesh, which are of different diameters mesh tube rolled in a roll, cut of desired length for surgical dressings fixation to the wound; and

where hydrophilic bandage has the ability to absorb water and is available in two versions: sterile and non-sterile (4-20 cm width); and

where starched bandage made of starched gauze or organza and is used as a reinforcing material over hydrophilic bandages (directly on the wound can “adhere” and damage the skin at the bend); and

where adhesive bandage containing zinc is a regular bandage applied with a thin layer of paste containing glycerin, gelatin, sodium chloride, zinc oxide, and which refers to medical DF shrinking when drying and dressing becomes very tight and used where necessary to avoid swelling of tissues, such as inflammatory skin diseases; and

where nonsterile gauze bandages produced 10 m×16 cm size, 10 m×10 cm, 5 m×10 cm, 5 m×5 cm, 5 m×7 cm, 7 m×10 cm, 7 m×14 cm, 7 m×7 cm both in secondary and individually packed; and

where dressing devices produced of dressing materials and finished products are intended for use and include groups such as bandages, packets, napkins, plasters, tampons, aerosols (sprayed foams and films), wound coverings; and

where the group of wipes distinguish itself napkins, dressings (e.g., gauze, napkins) and medical napkins (e.g., napkins “Koleteks™”); and

where gauze napkins are double layer of gauze cuts of size 16×14 cm, 45×29 cm and so on, and that both sterile napkins are available in pack of 5, 10, 40 pcs., nonsterile—100 pcs.; and

where medical napkins that are compositional therapeutical form, is a medical biopolymer on the substrate (usually cloth) immobilizing pharmaceutical substance or cloth impregnated with pharmaceutical substance; and

where napkins “Koleteks™” are a compositional dressing device that is a layer of special textile material as a carrier for biopolymer (sodium alginate), which has a therapeutic effect, or cloth framework that soaked pharmaceutical substance, and containing hemostatic, inflammatory, analgesic and wound healing agents (furagin, chlorhexidine, propolis, sodium alginate, urea, metronidazole) in various combinations, and are intended for use as a therapeutic and prophylactic agent for primary closure of injured tissues, stitched wounds, to close infected and granulating wounds, trophic ulcers, burns, bedsores, and are packed in original packaging in a sterile (inside) paper packet and secondary packaging—cardboard boxes; and

wipes hydrogel “Koleteks™-AKL” with sodium alginate, ε-aminocaproic acid and lidocaine for use as bleeding control agents (during scheduled and emergency surgeries in emergency care of injuries involving external bleeding in industry, in everyday life, in road traffic injuries, etc.); and

where packages are ready bandage dressings for applying to the wound to prevent it from contamination, infection and blood loss, and where the individual sterile dressing consists of hydrophilic bandage (7 sm×5 m), cotton pads (13.5×11 sm) which can be turned up to the top of the bandage, and pin to fasten the ends of the bandage; and where cotton-gauze pads soaked with a solution of corrosive sublimate, and where these packets distinguish two types—small and large, in which one or two pads (one turned up to the top of the bandage, the second—free), and where individual dressing packages are made so to a constant wearing sterility was not raised, and where, if still containment is broken, the core of the package is sterile; and

where these pads are made such that little stick to the wound (insignificantly adhere to exudating wounds); and

where tampons dressings are a small piece of cotton or linen dressing used to close wounds or sores or for bleeding control (especially during surgery to remove blood from the dissected vessels); and

where plasters used as dressings according to aim of fixing and covering plasters, and where covering plasters may further comprise a pharmaceutical substance, and where fixing plasters used in surgery and traumatology for securing dressings; and where the coating plasters are used in dermatology for the treatment of some diseases or mechanical damage to the epidermis, and where usually plaster dressings combine codenamed “adhesive plaster” and that appearance divided into strips and tapes and usually one side with adhesive layer; and among which the coating plasters on sticky side is attached gauze pad that is impregnated with a pharmaceutical substance (e.g., plaster bactericidal), and are given as commercial products, including “leucoplasts”, “Siofaplast”, “Trikoplast”, “Santavik” etc., and which additionally can be in form of perforated plasters on paper under the trade names “Leykopor” “Betabant” et al., and in particular, are plasters “Uniplast”, including: fixing adhesive medical tape with dimensions 500×10 cm, 500×1.25 cm, 500×2.5 cm, 500×0.5 cm; which is available in rolls with protective coating and smaller on reels, and where the tape consists of viscose elastic fabric and nonwoven fabric adhesive, and where strips “Uniplast Plus” secure reliable bandage fixation, protects the wound from germs, do not cause allergic reactions and skin irritation, do not leave marks on the skin and clothing; and

where plasters are manufactured in various sizes and configurations, including rectangular or round shape, on fixing sticky tape with or without perforations in packs of 8, 10, 20 pcs. of one size and sets of 10, 16, 24, 30 pcs. products of different shapes and sizes;

and where among of dressing strips are waterproof, hypoallergenic, elastic (suitable for use in the joints area), and where a series of patches of antimicrobial action of Band-Aid is produced by “Johnson & Johnson”, which are made of non-woven material that does not stick to the wound, contains antiseptic benzalkonium chloride, is transparent and adhesive coating fixes plaster on the skin without causing irritation and dimensions of which are 7×2 cm, 4×1 cm, 4×4 cm, and are available in packs of both sets of different sizes (24 pcs.), and among of them antiseptic waterproof, antiseptic textile are suitable for protecting wounds on bends; and

where medical sponges are hemostatic, gelatinic, collagenic, alginatic; and

where medical sponges comprise therapeutic dosage or non-dosage form, comprises a porous mass of different sizes and shapes, containing pharmaceutical substance and excipients (mainly plastics), and in form of plates of different sizes (50×50, 100×100, 90×90, 240×140 mm et al.), and which are mostly made of leather or tendons of cattle, algae and produced in sterile packaging; and

where hemostatic sponge made of human plasma with the addition of calcium chloride and ε-aminocaproic acid presented by dry, porous substance, is white with a yellowish tint, for topical administration, and gradually dissolves in wound, and containing thrombin, fibrin, ε-aminocaproic acid, and supplied in vials, and may be produced of collagen; and

where absorbing gelatin sponge is a sterile hardened foam soluble in water and resorptable in tissues, and is designed for bleeding control in surgery, and gelatin sponge-starch is type of gelatin sponge used for the same purpose; and

where collagen sponges are sterile porous plates produced of collagen, and resorptable in tissues, having hemostatic and weak adhesive properties, so widely used for wound coverings, and are often combined with various natural polymers and medicinal substances (e.g., chitosan, pectin, antibiotics, etc.) that allow significantly improve their consumer properties; and

where commercially available sponges include the sponge “Alhypor” made of alginate and is sterile and could be applied to the wound and absorbs discharge from the wound and eventually dissolves and containing pharmaceutical substances been shown to facilitate healing, and applicable for the treatment of venous ulcers, bedsores, and due to complete resorption can be used during operations on internal organs; sponge “Alhymaf”, which is a modification of “Alhypor”, and another set containing antiseptic substances, and rapidly facilitating healing of wounds; and

where the wound covering, primarily designed for the treatment of chronic wounds and their composition and varieties depend on the type and stage of wound treatment process (main stages of treatment: cleaning, removal of organic substances, granulation, vascularization, epithelization), and are produced in the form of coatings (alginate, sponge, hydrogel and hydrocolloid) as dressings designed to absorb wound exudate and control wound hydration, and where a wound covering used as permeable films and membranes; and

where the sucking coatings (adsorbing) perforated solve the problem of mesh dressings adhering when drying to wounds with exudate mild to moderate quantities; and where, as one example of the implementation of industrial coatings, Austrian firm “NYCOMED” produces adsorbing wound covering “Tahokomb”, intended for hemostasis and tissue adhesive, especially during surgery of parenchyma of various organs (liver, spleen, etc.), gynecology, urology, vascular surgery, trauma, etc., and where “Tahokomb” is a collagen plate coated with a special fibrin adhesive, containing fibrinogen, thrombin, riboflavin, etc., and where imposed whereon the wound “Tahokomb” plate undergoes resorption in the body for 3-6 weeks and is produced in a sealed package and is used in the harsh sterility and size of the plates which make up 9.5×4.8×0.5 cm; 1 pc.

where the wound films, which are usually sterile perforated sheets of different colors (yellow, dark blue, colorless, etc.) depending on antiseptics, within their structure, and presented of different types, including “Aseplen”, “Vynyplen” “Byokol-1”, “Vasoderm-S”, and where the polyvinyl alcohol aseptic film “Aseplen” is intended for the treatment of infected wounds, burns of I-II degree, temporary closure of transplanted skin autografts and donor sites, and available in three versions: with dioxydine (“Aseplen-D”), iodine (“Aseplen-I”), with catapole (“Aseplen-K”) and is hydrophilic, easily modeled on the wound, and through perforated holes not prevent the outflow of wound secretions and provides prolonged antimicrobial effect, easily removed from the wound surface, creating a delicate crust and favorable conditions for regeneration processes in the wound, prevents the development of infectious complications; and

where perforated polyvinyl alcohol film “Viniplen” is intended to treat wounds donor sites at dermatomal leather plastics and can also be used for temporary closure flat wounds of different etiology, cosmetology, etc., and is non-toxic, less time heal wounds, to avoid tanning treatment of disinfectant solutions without traumatic wound and has good draining properties; and where the film with petroleum jelly “Vasoderm-S”, which is made from specially treated cotton fabric and impregnated with a neutral ointment containing anhydrous wax, liquid petroleum jelly, fish oil, Peruvian balsam, and which is used for treatment of fresh and weeping sores, burns, detachment nails, ulcers, phimotic operations, in the transplantation of skin, plastic surgery and various skin lesions, and is not adhere to the wound, absorbs excretion, improves granulation and epithelization, prevents secondary infection, has antiseptic action; and

where biological wound covering “Biokol-1” represented by transparent, flexible, porous film that reliably self-locked on the wound, helps stimulate regeneration, leading to faster wound healing, and is absolutely atraumatic, having analgesic effect and is used to treat burns, trophic ulcers, donor sites and protection autografts; and

where bandages are the fabric, covering the wound or part of the body to protect from external influence and promote healing; and

where aseptic bandages made of sterile dressings (one or two cotton-gauze pads, gauze bandage and latch) and are intended to protect against microbial infection and other contaminants wound surfaces, and

where synthetic bandages “Elafoam” intended for treatment of various wounds, including burns, available in single packs and are sterile and their use can reduce up to half the number and duration of dressings; and

where various bandages produced as a kind of wound coatings that absorb exudates and show therapeutic effect by the content of various medicinal substances (suction, deodorizing, primary viscose, povidone-iodine et al.), and containing immobilized enzymes, such as “Dalcecs-tripsin”, “Lax-tripsin”, “Dalcecs-Kollitin” and having cellulose or polycaproamide carrier with immobilized proteolytic enzymes, including trypsin or lysozyme, collinite and used in surgery for the treatment of purulent necrotic wounds at the stage of hydration, and also pressure sores, ulcers of various etiologies, burns http://www.znaytovar.ru/s/Klassifikaciya_i_xarakteristika2.html).

However, these hemostatic devices agents are unsuitable for use in combat conditions for control bleeding to stop the heavy bleeding, because in such conditions as first aid, mostly provided by 1) not professionals or itself, 2) in a very short time, 3) disadvantaged conditions 4) with an additional risk to those who care. Also, these tools are not suitable for use as their presentation, storage, poor hemostatic effect, the need to prepare solutions immediately before use and medical supplies are inadequate to the means for a quick stop massive bleeding in large and medium damages (including gunshot and projectile injury), mostly on the battlefield at the point when the self, mutual and unskilled first premedical and medical care. Beside of the limited time and opportunities for medical qualified assist the wounded there is the issue of transportation to the place of professional medical care. During transportation, due to the impossibility to ensure complete comfort of wounded and its careful transportation possibly the opening of bleeding, leading to further blood loss.

### LIST OF FIGURES

FIG. 1-5. Mesh structure of carrier.

FIG. 6. Structure of substrate and device “napkin”.

FIG. 7-14. “Two-dimensional” devices (substrates).

FIG. 15. Window-form bandage with additional bandages and fastenings and tampons.

FIG. 16-17. Tampons.

FIG. 18-21. Narrow tampons “sticks” and “cord” with additional applicators.

FIG. 22-24. Tampons “pockets” and “pads”, including with additional hemostatic devices inside. Tampons “pockets” and “pads”, including with additional hemostatic devices inside.

FIG. 25. Hemostatic devices, comprising gel-like hemostatic agents, corrugated bandage and syringe.

FIG. 26. Various variants of syringe filling with hemostatic devices and hemostatic composition.

FIG. 27. Hemostatic devices, comprising hemostatic tampon-like (in particular compressed) and hemostatic composition, its combinations, corrugated bandage and syringe.

FIG. 28. Hemostatic device “Syringe”, filled with compressed hemostatic devices, in particular connected balls.

FIG. 29-30. Spray and tube.

FIG. 31. Examples of radiographic hemostatic composition and device.

FIG. 32. Shown influence of composition of the hemostatic composition on time of fibrin thread (filament) formation (start-to-finish time of whole process).

### BRIEF DESCRIPTION OF THE INVENTION

The basis of the group of inventions is the problem by means of selecting the optimum qualitative and quantitative composition of hemostatic agents (HA) to create a composition of hemostatic agents (CHA), i.e. hemostatic composition, suitable for combination (connection) with the selected container (substrate) and manufacturing of hemostatic agent to stop the heavy bleeding for use on the battlefield and disaster medicine, and other conditions that require rapid assistance by non-specialists.

The basis of the creative idea was the idea of developing a composition of hemostatic agents (CHA), i.e. hemostatic composition, capable to reliably bind (obduce) solid dusty particles mainly of inorganic hemostatic agents and thus prevent dust formation, and where the selected container (substrate) designed for combination with said composition, should have such physicomechanical, physicochemical properties to ensure its suitability for the composition of hemostatic agents for obtaining of hemostatic device with suitable properties for use as a hemostatic device for the control of heavy massive bleeding on the battlefield and in disasters medicine.

As a result, a series of experiments, it was found that the hemostatic composition comprising a water-retaining, dust-suppressing binder, inorganic and organic hemostatic provides the following features of the said hemostatic composition (or hemostatic device): reducing the time of commencement and completion of thrombus formation, prevent drying-up of the clot, increased moisture retention in the clot. Because the clot formation begins at 30^{th} second after application of the hemostatic device to a great extent bleeding stopped. Because of at the end of 2^{nd} minute clot formation ends there is a possibility for quick transportation the wounded to a safe place where he can obtain a quality medical care. The composition of hemostatic agents, i.e. hemostatic composition, consisting of water-retaining hemostatic agent, dust-suppressing binder hemostatic agent, inorganic hemostatic agent, organic hemostatic is made in the form of powders, solutions, suspensions, foams, paste, gel.

The composition of hemostatic agents, i.e. hemostatic composition, characterized in that the said hemostatic composition is configured such that it comprising simultaneously several components selected from the following groups 1) group of water-retaining hemostatic agents, 2) group of binder dust suppression hemostatic agents, 3) group of inorganic hemostatic agents and 4) group of organic hemostatic agents, having different nature and different influence on factors of the blood coagulation system, and that at least part of every one of the said hemostatic agents, can contact with blood when treating bleeding, and due to this and also with that the said hemostatic composition is configured such that its qualitative and quantitative composition provides a synergistic acceleration of formation of fibers of fibrin and consequently the manifestation by these agents, that are part of the composition, a synergistic hemostatic effect that accelerates blood clotting.

The hemostatic devices, characterized in that the said device is configured such that when treating wound bleeding, application of the device provides that at least a portion of the water contained in the blood is adsorbed by the substrate, that causes increasing of density of blood and assists in accelerating blood clotting.

The hemostatic devices, further characterized in that the said device is configured such that the said hemostatic composition containing simultaneously water-retaining, binder dust suppression, inorganic and organic hemostatic agents, having different nature and different influence on factors of the blood coagulation system, is distributed on the developed surface of the said substrate, that provides multiple contact with blood of every deposited on this surface of the said hemostatic agent, and due to this and also with that the said hemostatic composition is configured such that its qualitative and quantitative composition provides a synergistic acceleration of formation of fibers of fibrin and consequently the manifestation by these agents, that are part of the composition, a synergistic hemostatic effect that accelerates blood clotting.

### DETAILED DESCRIPTION OF THE INVENTION

Blood, which is a liquid connective tissue, is a polydisperse system—suspension of blood cells (erythrocytes, platelets, white blood cells) in plasma (where proteins form a colloidal solution, but other organic substances and inorganic salts form a true solution). Blood plasma is 5% of body weight consists of water (90%), proteins (7.8%), among which are albumins (70%), globulins, fibrinogen and blood coagulation factors (II, V, VIII, X), fats (0.8-1%), glucose (0.12%), urea and uric acid (0.5%), minerals (0.9%), mostly NaCl, salts of Ca, K, Mg. This concentration is maintained at a constant level. Proteins provide a blood viscosity, latter increases with water loss, which can lead to blood clots formation. The buffer system of blood (pH=7.2-7.4) for 15% provided by blood proteins (by zwitter-ion balance between their amino and carboxyl groups), and for 85% by weak acids and their salts (mainly carbonate and phosphate buffers).

From this it is clear that in addition to launching a native blood clotting mechanism, any action leading to blood concentration (increase of viscosity) by rapid removal of water, binding (removal) of inorganic salts and drastic changes in pH inevitably lead to proteins coagulation therefore facilitate hemostasis of blood, except coagulation of blood clotting factors and removal of calcium ions.

The natural process of blood clotting (hemostasis) is an enzymatic chain process in which on the matrix of phospholipids, which are debris (fragments) of membranes of damaged cells, walls of blood vessels, tissues, blood cells, and get into the blood, sequentially are activated clotting factors and formed their complexes. Phospholipids of cell membranes act as catalysts of interaction and activation of clotting factors accelerating the progress of the hemocoagulation process.

Since in our case the stop severe bleeding we are interested in the mechanism of hemostasis (which takes place in three phases) to the outside (tissual) rather than internal (intravascular) blood coagulation system. External path runs by tissue thromboplastin (phospholipids) that stands out from the walls of damaged blood vessels and surrounding tissues. In the I phase the tissual platelet prothrombinase (factor V) is rapidly (5-10 s) formed, and the impetus for the formation of which is the appearance of tissual thromboplastin in blood. At once with the advent of the tissual platelet prothrombinase on the II phase as a result of prothrombin sorption on the tissual platelet prothrombinase surface prothrombin it quickly (2-5 s) transforms (under assistance of factors V, X and Ca²⁺) into thrombin. At the phase III the process of conversion of fibrinogen to fibrin occurs, which takes place in three stages: on the first stage under the influence of thrombin with fibrinogen forms sol like fibrin monomer; on the second stage under the influence of Ca²⁺ ions occurs fibrin monomers polymerization and fibrin polymer formed (soluble fibrin “S”); on the third stage under assistance of factor XIII and fibrynase (fibrin stabilizing factor) of tissues, platelets and red blood cells produced final or insoluble fibrin “I”.

Fibrynase (fibrin stabilizing factor) provides formation of strong peptide bonds between neighboring molecules of fibrin polymer that cements fibrin, increases its mechanical strength and resistance to fibrinolysis. The formation of fibrin completes the formation of blood thromb.

It is clear that at severe bleeding thromboplastin content in different areas of bleeding is different (it is evenly distributed throughout the volume of blood that flows) resulting processes occurring in different areas at different speeds.

Therefore, when creating composition hemostatic agents and devices based on it were taken into account the features of hemostasis mechanism.

As a result, a series of experiments, it was found that the hemostatic composition comprising a water-retaining, dust-suppressing binder, inorganic and organic hemostatic provides the following features of the said hemostatic composition (or hemostatic device): reducing the time of commencement and completion of thrombus formation, prevent drying-up of the clot, increased moisture retention in the clot. Because the clot formation begins at 30th second after application of the hemostatic device to a great extent bleeding stopped. Because of at the end of 2 minutes' clot formation ends there is a possibility for quick transportation the wounded to a safe place where he can obtain a quality medical care. The composition of hemostatic agents, i.e. hemostatic composition, consisting of water-retaining hemostatic agent, dust-suppressing binder hemostatic agent, inorganic hemostatic agent, organic hemostatic is made in the form of powders, solutions, suspensions, foams, paste, gel.

In contact with the blood each of performs its role (function). Given that blood is a liquid consisting of formed elements (erythrocytes, platelets, white blood cells etc.) dispersed in plasma containing different proteins (albumin, fibrinogen, etc.), amino acids and electrolytes (sodium, calcium salts) and maintained homeostasis of its components, at constant level values of pH (pH=7.2), ionic strength, viscosity. At the same time, many components contained in blood plasma (ions of calcium, fibrinogen, etc.) and erythrocytes (platelets) are a key factors for blood clotting, participating in different processes that occur during hemostasis. Therefore, it is clear that additional (artificial) violation of harmony in this sophisticated system such as blood characterized not only by its biochemical composition and biological (biochemical and physiological functions), but certain physical, physicochemical and chemical properties, can be caused in particular by the increase in blood viscosity (for example by the use of water-retaining agent, which may account for the intense suction of water (moisture) to concentrate the blood. This small, at first glance, the effect can cause varying the pH of the blood, its viscosity, ionic strength. In turn, these changes may lead to changes in the native conformation of proteins, including enzymes—factors of blood clotting, thereby activating and accelerating the process of blood clotting. In addition, the concentration of blood should facilitate and accelerate the fibrin thread formation and the formation of these spatial grid to which platelets stick. These reasons can cause destruction of the native tertiary structure of blood proteins and their coagulation, which would also contribute to hemostasis (unless coagulation of proteins factors of blood clotting).

It is also clear that the chemical compound, matter or material seriously affecting the ionic strength of blood plasma by reducing the content of electrolytes in it (or vice versa due to their substantial increase) or due to changes in pH of blood because of binding of low molecular ligands (amino acids, carboxylic acids) or vice versa, its (blood) saturation with ligands, which in addition to effects on pH can also bind to blood proteins, causing changes in their native conformation and coagulation. To these compounds, which can seriously affect the ionic strength of blood plasma belong inorganic hemostatic agents, including zeolites, activated carbon modified, some clays, ortotitanic acid etc., and various organic hemostatic, in particular, carboxymethyl cellulose, polyacrylic acid, alginic acid, etc. To these compounds, which can have a significant impact on blood pH changes belong particularly, among the hemostatic inorganic, including various modified clay mineral oxides of acidic or alkaline form surfaces, etc., and among organic hemostatic, including carboxymethyl cellulose and its salts, polyacrylic acid, polyvinylpyrrolidone, chitosan etc.

These can perform multiple functions simultaneously. For example, water-retaining (e.g., carboxymethyl cellulose, alginic acid and salts thereof) can simultaneously perform the function of binding dust-suppressing hemostatic agent, and organic hemostatic agent. Inorganic hemostatic in addition to features of significant extraneous surface for the liquid blood, accelerates the launch of blood clotting system in different (combined) mechanisms through chemical features (for example, if it contains calcium) due to the large surface contact with the liquid blood can in many points of “blood volume”, not only from the surface of said “blood volume” inwardly this volume (as is the case in the absence of hemostatic agent) run mechanism of blood clotting and especially the mechanism of fibrin fibers formation, which fetus are formed on the surface of inorganic particles of hemostatic agent, and quickly spread into the “blood volume” in all directions and thus form a mesh of fibrin fibers, which stick platelets and gradually formed a dense homogeneous clot.

As noted above, the effect of hemostatic on hemostasis can be as positive but negative as well. Therefore, it is important to choose not only the best quality composition of the hemostatic, but its quantitative constitution, especially taking on account that the hemostatic agents of such hemostatic composition have simultaneously multiple hemostatic functions, as discussed above. Amount of relevant hemostatic in the hemostatic composition should be sufficient, but not excessive, for the manifestation of the most positive impact on hemostasis, while the negative effects should be minimized. Besides excess of a hemostatic e.g. dust-suppressing binder, can significantly reduce or even reverse the positive effects, e.g. inorganic hemostatic agent, by blocking surface of the particles of latter and therefore the obstructing blood access to them. This should also consider the possibility of chemical and physicochemical interaction between a hemostatic comprising the hemostatic composition. The result of such interactions can be cooperative (synergic) or anti-cooperative (anti-synergic) hemostatic effect of hemostatic in the hemostatic composition.

The solution to this problem was obtained as a result of the experiments, the results of which are shown in FIG. 32. It was unexpectedly found that a combination of known hemostatic agents together in the same hemostatic composition (device) gives a positive result (improved hemostatic effect, for example, of two components (hemostatic agents), compared to the effects of individual components.

To compare hemostatic effects of different hemostatic agents—water-binding (dust suppression), inorganic and organic, was elected one of the key processes of hemostasis, namely the formation of fibrin fibers (filaments) and used the so-called “Model of fibrinous threads” ( [symbol] [symbol] [symbol] / [symbol] [symbol] . [symbol] [symbol] . — [symbol] .: [symbol] , 1994.—C. 272-283. [symbol] [symbol] [symbol] / [symbol] . [symbol] . [symbol] .—M.: [symbol] , 1984.—C. 217-226. [symbol] [symbol] [symbol] : B 3 Tomax/Πep. c aH [symbol] ./ [symbol] . P. [symbol] [symbol] ┌. [symbol] .—M.: [symbol] , 1996.—T.2.—C. 430-439. [symbol] Φ. [symbol] . [symbol] [symbol] [symbol] [symbol] [symbol] . — [symbol] : [symbol] , 2002), with which fixed time, which is needed for starting of fibrin thread formation process since fresh blood contact with study testing material and the time for which the process is completed, and the time interval in which the process proceeds erythrocyte agglutination Some of the results of these experiments, as example are given in FIG. 32.

Samples were prepared as follows: first preparing a solution or suspension of hemostatic, mixed them and receive hemostatic composition as a solution or suspension consisting of two, three, four, etc. components. Then these compositions result into interaction with various types of substrates—woven, non-woven, knitted, using any arbitrary manners and obtain dry or semi dry samples of hemostatic devices, consisting of substrate, which impregnated with one, two, three, four, etc. hemostatic. Then obtained as described above multicomponent solutions (or suspensions) of hemostatic, i.e. hemostatic compositions, or as described above hemostatic devices result into contact with fresh blood.

### Brief Description of the Methodology of the Experiment.

Samples were prepared as follows: take samples of fresh blood (the same donor) each time fresh puncture, drop of blood applied to flat substantive piece of glass with laid on top of him material and covered on top another objective lenses, ready to sample immediately put in against lenses microscope (for observing nature agglutination) and thus already observed interaction with the sample drop of blood hemostatic material (the period from the beginning of blood collection before the observation is 10±1 sec). This blood with that of the fences used to experiment with fibrin fiber. For this drop of blood placed on a glass slide with the deepening of the sample placed in its material. Immediately specialist special moves needle controls the appearance of fibrin fibers deep in the blood, which is adjacent to the material and records (on a timer) start the formation of fibrin fibers fibrinogen and end time of the formation of fibrin fibers in a sample of blood (drop) on the glass or on sample material, which is on the glass).

Agglutination of red blood cells under a microscope and the appearance of fibrin on lenses were simultaneously observed by two specialists on the identical samples of the same material and of the same blood. Agglutination of red blood cells was observed with microscope (Carl Zeiss company) in real time, changes in time were recorded with a timer. The procedure in all experiments was standard (the same).

In FIG. 32 shown results of experiment with formation of fibrin thread formation in donor blood samples at different hemostatic materials (lines on the graph is connected points indicating time (s) when starts (t1) and ends (t2) fibrin thread formation for samples: No 1 (glass surface); No 2 (non-woven substrate); No 3 (knitted); No 4 (woven substrate); No 5 (HSA http://www.celoxmedical.com/Celox™); No 6 (HSA [U.S. Pat. No. 7,604,819 B2, U.S. Pat. No. 8,114,433 B2, U.S. Pat. No. 8,257,732 B2, U.S. Pat. No. 8,383,148 B2, U.S. Pat. No. 8,343,537 B2] QuikClot™); No 7 (polyethylene glycol); No 8 (polyvinyl alcohol); No 9 (sodium alginate); No 10 (chitosan); No 11 (glycerol); No 12 (carboxymethyl cellulose (CMC)); No 13 (carboxymethyl cellulose sodium salt); No 14 (polyvinyl pyrrolidone); No 15 (bentonite); No 16 (kaolin); No 17 (titanium dioxide); No 18 (barium sulfate); No 19 (gallic acid); No 20 (polyacrylic acid); No 21 (tannin); No 22 (bentonite-CMC); No 23 (bentonite-tannin); No 24 (CMC-tannin); No 25 (kaolin-chitosan); No 26 (kaolin-sodium alginate).

For comparison, as control in FIG. 32 shown temporal characteristics for the fibrin thread formation on glass surface without contacting blood drop with hemostatic agents or hemostatic devices (sample No 1). Since for correct comparison of hemostatic agents to each other hemostatic agents planned to use as a hemostatic device (hemostatic agent with the same carrier) various textile carriers were previously tested (woven, non-woven, knitted, etc.), testing results of some of them for example and comparison shown in FIG. 32 (samples accordingly No 2, No 3, No 4). As seen from the comparison samples No 2, No 3, No 4 with a sample No 1 (FIG. 32) carrier itself (but differently, that maybe due to the different structure of substrate) slightly accelerates fibrin thread formation and most of all it relates to nonwoven textiles (sample No 2). This is likely due to the launch of native blood clotting mechanism to a foreign body, and acceleration of the process of fibrin thread formation for these samples compared to glass (sample No 1) possible associated with concentration of different coagulation factors because of at least partial dehydration of blood (moisture adsorption by substrate) and consequently increasing its density and viscosity. This agglutination (clumping of red blood cells) on the substrate material such as sample No 2, beginning after 1 minute and ends at 3rd minute, but has uneven, focal (clusters) nature. Based on above for the manufacture of other samples as a substrate were chosen one from group of the non-woven materials (sample No 2). For comparison were used the best of known for today hemostatic products—corrugated (z-folded) field gauzes (bandages) of third generation containing chitosan (http://www.celoxmedical.com/Celox™) (sample No 5) and kaolin [U.S. Pat. No. 7,604,819 B2, U.S. Pat. No. 8,114,433 B2, U.S. Pat. No. 8,257,732 B2, U.S. Pat. No. 8,383,148 B2, U.S. Pat. No. 8,343,537 B2] QuikClot™ (sample No 6).

It was found that temporal characteristics of the fibrin thread formation for hemostatic agent (http://www.celoxmedical.com Celox™) (sample No 5), made of thick non-woven substrate and chitosan, are virtually identical to the characteristics of the nonwoven carrier (sample No 2), but agglutination for the sample No 5 (unlike sample No 2) goes fast (1-2 minutes), evenly and with high density.

Unlike the HSD (http://www.celoxmedical.com/Celox™) (sample No 5) HSD [U.S. Pat. No. 7,604,819 B2, U.S. Pat. No. 8,114,433 B2, U.S. Pat. No. 8,257,732 B2, U.S. Pat. No. 8,383,148 B2, U.S. Pat. No. 8,343,537 B2] QuikClot™ (sample No 6), also made of non-woven substrate and kaolin, significantly accelerates fibrin thread formation compared to blood samples in vitro (sample No 1) and carrier (sample No 2), and unlike these samples agglutination in case of sample No 6 (begins at 60 s ends-120 s) runs quickly and evenly, with a high density but begins and runs mainly along the fibers, possibly due to the action of kaolin particles attached to fibers.

Further, for example in FIG. 32 shown results of investigations of some of the samples made from non-woven substrate and hemostatic agents (water-retaining, dust suppression binders, inorganic and organic) taken separately and in various combinations (pairs) with each other. Samples made of water-retaining hemostatic agents—polyethylene glycol (sample number 7), polyvinyl alcohol (sample No 8), sodium alginate (sample No 9), chitosan (sample No 10), glycerin (sample No 11). As shown in FIG. 13 these samples have different effects on the rate of fibrin thread formation in comparison, for example, with a substrate (sample No 2), and most of them, except for the sample No 9, containing sodium alginate, even hamper the process. This is possible due to the blocking of micropores existing in the substrate and with capillary forces, the blood is absorbed in the substrate. In the case of alginate, which quickly swells and dissolves some acceleration of process may be due to its partial dissolving and direct contact with blood. It should also be noted that the sample No 10 contains chitosan, with characteristics very similar to the HSD http://www.celoxmedical.com/Celox™ (sample No 5). Thus agents, such as glycerol (sample No 11) significantly slowing down agglutination (90 s—start, 180 s—weak, loose agglutination across the surface of the sample).

For samples made of binding (dust suppression) hemostatic agents such as carboxymethyl cellulose (sample No 12), carboxymethyl cellulose sodium salt (sample No 13), polyvinylpyrrolidone (sample No 14), unlike the samples No 7-No 11 also having binding (dust suppression) properties, there has been observed some acceleration of the fibrin thread formation compared with substrate (sample No 2).

For samples made of inorganic hemostatic agents such as bentonite (sample No 15), kaolin (sample No 16), titanium dioxide (sample No 17), barium sulfate (sample No 18) unlike previous samples, there is a substantial acceleration (especially in the case of kaolin and bentonite) of the fibrin thread formation compared to carrier (sample No 2), which is probably due to the large surface of particles of these substances contacting with blood, and the differences in their behavior due to their different chemical nature. It should be noted that the sample containing kaolin (sample No 16) for time characteristics prevails even HSD [U.S. Pat. No. 7,604,819 B2, U.S. Pat. No. 8,114,433 B2, U.S. Pat. No. 8,257,732 B2, U.S. Pat. No. 8,383,148 B2, U.S. Pat. No. 8,343,537 B2] QuikClot™ (sample No 6) even more the HSD (http://www.celoxmedical.com/) Celox™ (sample No 5) that may be explained by the presence in the sample No 6 dust suppression agent, which somewhat reduces surface contact of clay with blood.

The samples made with organic hemostatic agents, such as gallic acid (sample No 19), polyacrylic acid (sample No 20), tannin (sample No 21), showing roughly the same characteristics (FIG. 32) as described above samples and with the water-retaining binding (dust suppression) hemostatic agents that are also organic and hemostatic agents. However, for example, for gallic acid (sample No 19) unlike carboxymethyl cellulose (sample No 12), impact on agglutination of red blood cells is different. Thus, sample No 12 with CMC accelerates agglutination (starts 40 s, the ends 150 s), but it has a focal, uneven, with a significant number of voids (areas without agglutination) and sample No 19 with gallic acid positively affects on speed (the beginning of 60 s, 90 s—total agglutination along the fibers, 120 pp—complete agglutination across the surface of the sample, agglutination and a uniform, dense nature. Thus, influence of e.g. CMC and tannin (samples No 12 and No 21 respectively) on time of fibrin thread formation similar to the effect of carrier material, since these agents also have water adsorbing effect that affects the concentration of clotting factors and increased concentration of its uniform of blood, but the CMC and tannin have different effects on agglutination.

Samples of hemostatic agents, which have been made from substrate and simultaneously two hemostatic agents unexpectedly demonstrated both positive and negative cooperative hemostatic effect (synergistic action). In particular, in FIG. 32, as example, shown results for some of these samples (samples No 22-No 25). As shown in FIG. 32, two-component hemostatic agents (sample No 23, containing bentonite and tannin, and a sample No 24, containing tannin and CMC) characterized by much more time of fibrin thread formation compared to monocomponent materials with bentonite, tannin and CMC (samples No 15, No 21, No 12, respectively), which may be explained by the slowing effect of tannin, due to the fact that tannin causes coagulation of albumin and other proteins of blood plasma, including coagulation factors (e.g. co-coagulation of fibrinogen with albumin). Due to this said hemostatic agents combined in a hemostatic device (sample No 23, No 24) showed anti-cooperative hemostatic effect. Negative cooperative (anti-cooperative, antisynergic) effect is presents in the process of agglutination. Thus, for the sample No 24 (CMC-tannin), agglutination starts at 40 second, but because of coagulation of albumin (perhaps due to tannin astringent effect) on the second minute released plenty of water micro drops, like blood foams and thus inhibits the fibrin formation and agglutination of red blood cells not resulted to formation of normal blood clot and causes gelatinization of the blood.

Unlike of samples No 23 (bentonite-tannin), No 24 (CMC-tannin), for two-component hemostatic devices (examples No 22 (bentonite-CMC), No 25 (kaolin-chitosan), No 26 (kaolin, sodium alginate) strong positive cooperative effect observed. In particular, in the sample No 22 (bentonite-CMC) fibrin thread formation begins (t1) at 35 second and at 105 second its formation (t2) completes. The cooperative effect for sample No 22 (bentonite-CMC) appears in the process of agglutination started (t3) at 15 second and is complete along the fibers (t4) for 30 second, and 60 second is full agglutination across the surface of the sample (t5). For comparison, the corresponding characteristics of agglutination for sample number 15 (bentonite) (t3=45 C, t4=60 seconds, t5=120 s) and No 12 (CMC) (t3=40 s, t5=150S; agglutination have focal pattern with a significant number of voids (areas without agglutination)) are much worse. For samples No 25 (kaolin-chitosan), No 26 (kaolin, sodium alginate) as well as for sample No 22 (bentonite-CMC), distinct cooperative effect observed, for example, evidenced by comparing the start time (t1) and end (t2) of fibrin thread formation in sample No 25 (kaolin-chitosan) ((t1=75 s; t2=150c) with sample No 10 (chitosan) (t1=170 s; t2=240p) and No 16 (kaolin) (t1 p=110; t2=125 s) and sample No 26 (kaolin, sodium alginate) ((t1=35 s; t2=140 s) in accordance with sample No 9 (sodium alginate) (t1=140 s; t2=205 s) and No 16 (kaolin) (t1=110 s; t2=125 s).

Thus, obtained results indicate that due to cooperative (synergic) effects hemostatic agents can enhance the hemostatic action of each other. However, anti-cooperative effects can weaken the effect of certain hemostatic agents. Therefore, to obtain highly effective hemostatic device to control the severe bleeding it should to create a composition of hemostatic agents, i.e. hemostatic composition, with the optimal choice of qualitative and quantitative composition of components, functional properties of each of the agents not only complement each other but a combination of these agents in the composition would be achieved a new quality—a substantial increase of hemostatic action of the said composition and production of hemostatic device using it.

During the experiments it was found that initiation of clot formation and rapid, almost instantaneous initial moisture absorption is carried out by inorganic component, but this component has a drawback, which is relatively small dust and moisture binding. Organic components can provide significant moisture retention compared to their weight, but do not give an instant effect of moisture absorption. Moisturizers components ensure retention of a certain moisture level in absence of condensate. Certain level of humidity ensuring the mechanical properties of the material, its flexibility, prevents dust formation of inorganic component and also provides acceptable tactile properties of the material. It was also found that the differentiation of certain components is rather conditional, because certain components may belong to different groups, but their combination, based on their functional purpose in certain proportions compensates for deficiencies and increases positive qualities.

Thus, during experiments researchers has found that the optimal composition of hemostatic agents, i.e. hemostatic composition, should contain 4 groups of components:

water-retaining hemostatic agent,

dust suppressing binder hemostatic agent,

inorganic hemostatic agent,

organic hemostatic agent.

Water-retaining hemostatic is a compound selected from the group consisting of but not limited to polysaccharides and its derivatives, in particular, carboxymethyl cellulose, and/or salts and/or chitosan thereof and/or polyols including glycerol gums, in particular, locust bean, guar, xanthan, pectins and/or glycerol.

Binder dust suppressing hemostatic is compound selected from the group consisting of but not limited to: synthetic and/or natural, including modified polysaccharides, in particular, carboxymethyl cellulose, starch, agar-agar, gum arabic, dextrin, polyols in particular, glycerol sorbitol, xylitol, maltol polymer polyols, glycerol derivatives preferably, but not limited to, propylene glycols, glyceryl triacetates and/or cyclic alcohols, preferably, but not limited to, menthol, eugenol and combinations thereof.

Organic hemostatic agent is compound selected from the group consisting of but not limited to ε-caproic acid, tranexamic acid, amben, fibrin, polyphenols and/or its components, in particular, tannin and/or tannic and/or gallic and/or digallic acid and/or flavonoids, in particular, rutin and/or quercetin, and/or preferably selected from the group of water-soluble polymers, including natural polymers, including chemically modified natural polymers, preferably selected from the group of cellulose derivatives, gelatin, gelatinized starch, polyvinylpyrrolidone, dextrose, pectin, chitosan, agar-agar, gum arabic, collagen, polyvinyl alcohol, polyacrylic acid, and its salts silicone, polyvinyl acetate and/or group of polyols, preferably selected from the group glims, glycerol and its esters and/or plant extracts or vegetable extracts selected from the group but not limited to in particular nettle leaves (Folia Urticae), yarrow herb (Herba Millefolii); water pepper herb (Herba Polygoni hydropiperis), grass Persicaria maculosa (Herba Polygoni persicariae), Viburnum bark (Cortex Viburni opuli), arnica (Flores Arnicae) etc., and/or combinations thereof.

Inorganic hemostatic agent is compound selected from the group consisting of but not limited to water-insoluble and/or sparingly soluble oxides of natural and/or synthetic origin, chosen preferably, but not limited to the group consisting of oxides of titanium, silicon, aluminum, etc., selected preferably, but not limited to from the group consisting of attapulgite, kaolin, bentonite, etc. and/or its combinations and/or minerals selected preferably, but not limited to zeolites, including that might be part of clays, metal salts selected preferably, but not limited to, from the group consisting of calcium, barium sulfate, titanates, phosphates, glycerophosphate, etc.

Also, to ensure radiographic properties of hemostatic agent is proposed to add radiographic material. Preference is given to those radiographic substances having hemostatic properties. Such substances are preferably but not exclusively selected from the group consisting of barium sulfate, phosphate and titanate.

To visualize the hemostatic composition as any hemostatic agent or its fragments in X-rays, what is necessary to control the absence of said hemostatic composition or said hemostatic device in a wound after providing skilled care, including surgery, to prevent adverse effects caused “forgotten” hemostatic composition or device in the wound, hemostatic composition may contain at least one hemostatic agent having radiographic properties.

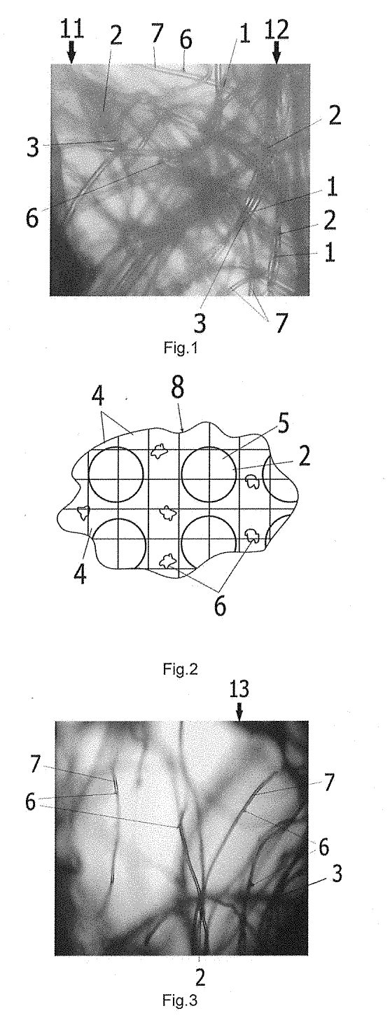

As one possible example of such hemostatic composition is composition of Example No 23, containing inorganic hemostatic agent—barium titanate, having radiographic properties. Next, as one of the possible examples (Example No 26) of such hemostatic composition containing inorganic hemostatic agent—barium sulfate, having radiographic properties. Made of such hemostatic compositions such as Example No 26, hemostatic device, is one of examples, such as hemostatic device “napkin” (16) (FIG. 6) in the radiographic variant (Device (170), X-ray photo is shown in FIG. 31, and that (device (170) consists of “composition” (171), having radiographic properties, and “two-dimensional substrate” (33) mainly, but not exclusively, square or rectangular or oval, and which radiographic materials (34) contained in the hemostatic “tracks” (6), using a mesh structure (8) (FIG. 2) “substrate” (33), which (FIG. 1, 3, 4) consists of yarns and/or fibers and/or strips that can be interconnected in any satisfactory way. Interlocking (1) and knots (2, 3) between components of mesh structure (8) “substrate” (33) allow to material to be flexible and keep constant the size of the holes (4, 5) between them (1,2,3). Yarns, fibers and strips of mesh material can be made of polymers (nylon, polyethylene, polypropylene, polyester, etc.) and/or glass fibers and/or organic matter (natural origin) (e.g., cotton, wool, silk, etc.) and metals and/or their combinations. “Substrate” includes special additional holes (5) and irregular grid cell (4) formed weave (1) of fiber and filamentary structures in knots (2, 3). Device (32) (FIG. 14) and device (170) (FIG. 31), is a cloth (16) (FIG. 6) where through said holes (4), and additional holes (5) and through the system of interlacing (1) and knots (2, 3) liquid (blood) can penetrate including due to capillary forces, and interact with the particles and molecules of hemostatic agents of mentioned “composition” (171) containing radiographic agent (34) and has radiographic properties, resulting in accelerated clotting. Thus, radiographic agent (34) is uniformly distributed in the “carrier” (33) and in X-rays such a “composition” (171), shown in Example No 26, and made hemostatic agent (170), has radiographic properties as shown in FIG. 31.

One possible example of hemostatic composition is shown in Example No 23, and which has radiographic properties in X-rays in FIG. 31 are arbitrary graphic (173), such as “X”, which is applied by writing with “composition” (171) of Example No 26 image (173) in a column “X” to “substrate” (33) in the form of device (174) “napkins”.

As seen from FIG. 31, both options of the possible hemostatic compositions of Examples No 23 and No 26, and hemostatic devices (170), (174) “napkin” described above, provide radio-opacity for these compositions and products in X-rays and the ability to detect the wound if they are left in the wound.

According to the requirements for such devices on undemanding to storage conditions and duration of storage hemostatic device placed into sealed packaging ensuring its sterility during shelf life.

The solution to the technical problem posed invention is to provide two objects: hemostatic compositions and “container” (“substrate”). Specifically, hemostatic composition as such and hemostatic device, which consists of said composition of hemostatic agents combined with the “container” (“substrate”), wherein “container” (“substrate”) contains said composition. It should be mentioned that “capacity” in the sense of the present invention is a significant set of different containers and substrates, which can be divided into two groups: the “two-dimensional container” (“substrate”) in which the value of a measurement to two other at least 10 times less than least two other and form these “containers” (“substrates”) is relatively simple (e.g. cloth, bandage, nonwoven fabric, etc.), and “three-dimensional”, in designs which can be quite complex, and there is no such value measurements as in “two-dimensional”.

“Container” (“substrate”) is agent or device which is material to any selected from the following group: cotton, silk, wool, plastic, cellulose, rayon; polymer (e.g., nylon, polyethylene, polypropylene, polyester (polyester, polycarbonate, etc.)), metal, glass, organic matter, a mixture of the above, woven, non-woven, water permeable and/or water impermeable.

Optionally, “container” (“substrate”) can be bandage, napkin, film that can be definitely folded and/or twisted and/or convoluted.

Optionally, “container” (“substrate”) can be in hermetically sealed packaging ensuring its sterility during shelf life.

“Container” (“substrate”) can be “three-dimensional”.

“Container” (“substrate”) can be a vessel containing the composition of hemostatic agents or said above hemostatic device or medication such as tampon, napkin, sponge at least part of, “container” (“substrate”) for the composition of hemostatic agents has a water impermeable shell. At least some container for the composition of hemostatic agents has a water impermeable shell, and the composition of hemostatic agents further comprises at least one component foam. At least part of “container” (“substrate”) for the composition of hemostatic agents may have water impermeable shell filled with a composition of hemostatic agents in the form of a gel, foam, paste. At least part of “container” (“substrate”) for the composition of hemostatic agents presented by flexible substrate e.g. gauze is fibrous and/or mesh and/or a structure with air holes, can incorporate composition of hemostatic agents in any sequence in any suitable way, particularly it impregnated with a solution and/or suspension composition of hemostatic agents used in spraying solution and/or suspension composition of hemostatic agents, and/or use “slot-die” process for applying the solution and/or suspension composition of hemostatic agents and/or using smearing of substrate with solution and/or suspension of the composition of hemostatic agents and/or using any combination of these methods, dried to the required moisture and applying at least portion of at least one side surface of substrate for complex of hemostatic agents with adhesive substance. Also, said device can be placed into sealed packaging ensuring its sterility during shelf life.

Hemostatic composition can have radiographic properties, can incorporate appropriate hemostatic agents having radiographic properties (radiographic agents) selected preferably, but not exclusively, from the group consisting of barium sulfate, titanate and phosphate.

As one of the possible options for providing radiographic properties of hemostatic composition and produced hemostatic device can be radiographic materials (36) selected from the group consisting of polymeric materials such as polypropylene, which is produced with the addition of radiographic agents (34), including barium sulfate and which are produced mainly in the form of fibers or tapes, which can be crushed into small pieces that can be added into the hemostatic composition to providing radiographic properties. Availability of radiographic material, e.g. said above polymer (made of polypropylene and e.g. barium sulfate) materials in the form of threads, bands, ribbons connected to the “substrate” (33) to produce hemostatic agent, such as device (32), as seen in FIG. 14 also provides a reliable indication of the presence of hemostatic device in the wound.

To visualize any hemostatic agent in X-ray of the “product” radiographic agent can be added (connect) directly to the material substrate. In FIG. 14 shown one of the variants of “device”—device (32), consisting of “composition” (6) and “two-dimensional substrate” (33), and preferably, but not limited to square or rectangular or oval, containing radiographic material (36) or radiographic agent (34) in the form of strips or ribbon, and which keeps the said “composition” (6) and includes radiographic components (34) or processed with radiographic agent (34) (e.g., drawn thread or dotted line (236) on the substrate (33) as shown in FIG. 14) or processed with thread (236), as shown in FIG. 14).

Meshwork (8) of “substrate” (33) in FIG. 2 consists of filaments and/or fibers and/or strips that can be interconnected in any suitable way. Interlocking (1) and knots (2, 3) between components of mesh structure (8) “substrate” (33) allow to material to be flexible and keep constant the size of the holes (4, 5) between them (1,2,3). Yarns, fibers and strips of mesh material can be made of polymers (nylon, polyethylene, polypropylene, polyester, etc.) and/or glass fibers and/or organic matter (natural origin) (e.g., cotton, wool, silk, etc.) and metals and/or their combinations. “Carrier” includes special additional holes (5) and irregular grid cell (4) formed weave (1) of fiber and filamentary structures in knots (2, 3). Device (32), which are listed as one of the possible options in FIG. 14, is a “napkin” (16) (FIG. 6), “substrate” (33) has mesh structure (8) (FIG. 2 and FIG. 1, 3, 4) through the holes (4) and additional holes (5), and through the interlacing system (1) and knots (2, 3) liquid (blood) can penetrate particularly due to capillary forces, and interact with the particles and molecules of hemostatic agents of said “composition” (6) or (171), resulting in accelerated clotting.

For application of device (32) to the bleeding wound, it should be extracted from sealed package and placed over of and/or inside the wound. The particles and molecules hemostatic agents (6) located in the “substrate” (33) contacting with the wound tissue and/or blood, liquid phase is adsorbed on the surface and by the volume of device (32), promoting blood clotting. Flexibility of “substrate” (33) allows a device (32) to take and maintain shape of the wound. Availability (FIG. 14) radiographic material (36) attached to the “carrier” as a strip or ribbon (236), made as one of the possible options, polypropylene and barium sulfate, and the use of such “substrate” (33) to produce hemostatic device (32) provides a reliable indication of the presence of hemostatic device in the wound.

The sealed package (not shown) provides a sterile hemostatic agent prior to its use.

In FIG. 13, shown device (35), to which added radiographic agent (34) for its X-ray visualization, in particular, as one of the possible options, barium orthophosphate and/or sulfate, directly into hemostatic composition (6) or with hemostatic composition (6). For example, device (35) may further include radiographic component (34), such as one of the possible options, barium sulfate, applied to the “carrier” arbitrary manner in the form of strips or ribbons (236) or in any other way, for example, as one of the options shown in FIG. 14 in the highlighted square frame of device (32).

As mentioned above to ensure efficient operation of hemostatic compositions and convenience of use and retention of its properties during shelf life and transportation said composition may be connected to the “container” (“substrate”) into one wherein those connected hemostatic composition and capacity are present a hemostatic device, and wherein capacity keeps composition of hemostatic agents. “Container” (“substrate”) can be “two-dimensional” particularly flat or “three-dimensional”.

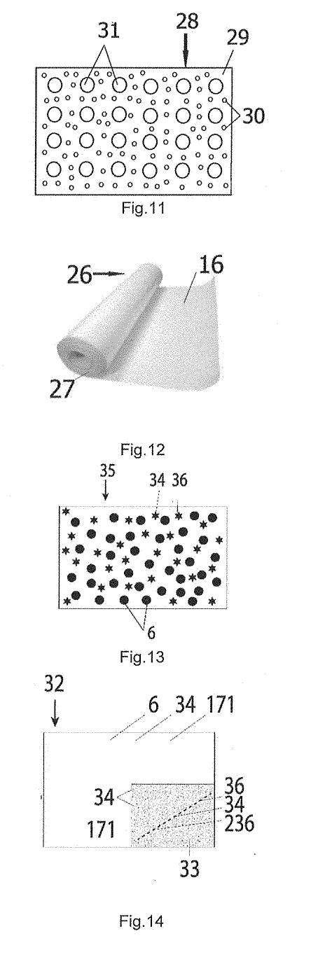

Hemostatic device made of “two-dimensional container”, (substrate), and “container” (substrate) itself, presents two-dimensional geometrical structure (shape), preferably, but not limited to rectangle (15), shown in FIG. 5, preferably, but not limited to flat, whose dimensions in length and width far exceed height, for example, having Length or Width to Height ratio at least 10 times, including those which for compact packaging (such as wipes, bandages) are folded (shown in FIG. 5 by axis (9)) or rolled/folded in a three-dimensional shapes, such as corrugations (shown in FIG. 8 device (21), FIG. 9 device (24)), rolls (shown in FIG. 12 device (26)) or for accessibility (e.g., cord, helix) with and/or as examples of two-dimensional, flat structures can be gauzes, bandages, napkins and more.

Such hemostatic device may be produced of container presented by agent or device which is material to any selected from the following group: cotton, silk, wool, plastic, cellulose, rayon; polymer (e.g., nylon, polyethylene, polypropylene, polyester (polyester, polycarbonate, etc.)), metal, glass, organic matter, a mixture of the above, woven, non-woven, knitted film to water and/or water impermeable. As one of the possible examples of the invention in FIG. 1 are shown two-dimensional device (its micrograph) made of two-dimensional capacity (10), which is a fragment (11) of nonwoven fabric composed of cellulose fibers and viscose. As noted above, even a material in itself reveals additional hemostatic effect upon contact with the blood and can be seen as a hemostatic agent (11).

Hemostatic properties of the “device” is an integral characteristic depending on the qualitative and quantitative composition of hemostatic agents comprising the “composition” and the material or materials from which made “substrate”, its macro- and microstructure, method of manufacture, etc. Based on the fact that the “substrate” should provide secure fixation and maintenance “composition” throughout the volume and/or on the entire surface of the said “device” and one, but not the only of its functions is to prevent the ingress of particles “composition” into wound, its mechanical separation from the “substrate” and the flexibility of substrate so that it can repeat the geometry of the wound, “substrate” comprising material selected preferably but not limited to the group comprising organic material of natural origin and products of its chemical modification (cotton, silk, wool, plastic, cellulose, viscose, etc.), polymers (such as nylon, polyethylene, polypropylene, polyester, polycarbon, et al.), metal, glass fiber, organic matter; mixtures of the above; woven, nonwoven, film water permeable and/or impermeable.

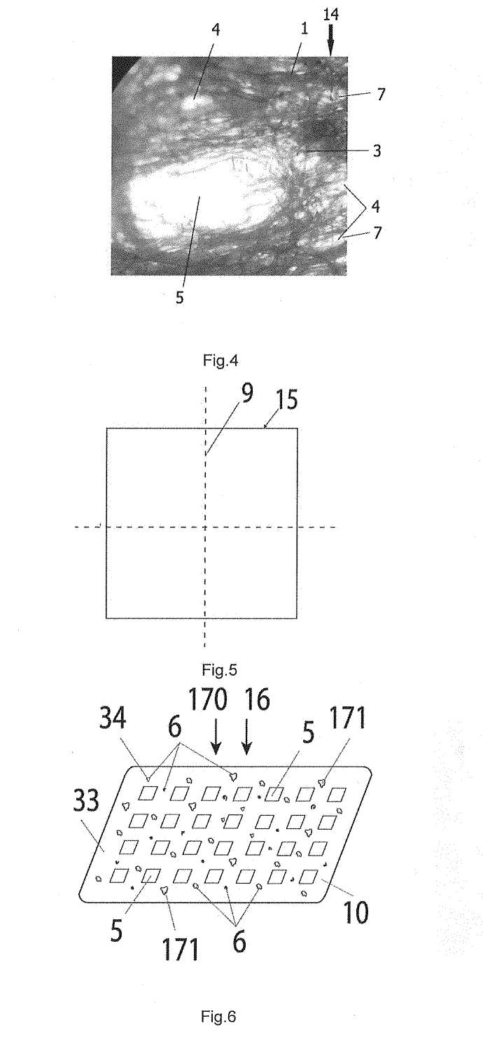

This “substrate” consists of filaments, fibers, strips, and combinations thereof. As possible options of such a “substrate” (10) shown in FIGS. 1-4, in photos of non-woven (12) can be seen (FIG. 1) its fibers twisted (interwoven) (1) along their axes and form filamentous structure, sometimes they intertwined in different directions and forming knots (2), sometimes twisted fibers of the filamentous structure and/or fiber (7) and (FIGS. 3, 4) forming knots (3). Such a structure of “substrate” provides its hygroscopicity, good moisture absorption and ability to maintain its flexibility, which allows it to draw up or roll up in any way for further packing in the “device”. Flexibility of “substrate” and made of it “device” allowing it to take appropriate wound to form and maintain it during use. The present invention is not limited to the use of woven or non-woven fibrous material as a material capacity, so, in particular, felt and similar to it outside the scope of this invention.

These knots (2) and (3) form a network structure (8), which is different in size and shape of the cell, namely holes (4), and for which are shown in FIG. 2 as a schematic representation of one possible example. In addition to the cells (pores (4)) such meshwork containing additional special pores (5) preferably but not limited to oval and/or square and/or rectangular and/or rhombic shape that are macropores compared to the size cells (4), and additionally provide the possibility of penetration of blood deep into the material and its contact with the hemostatic agents of the “composition”, and wherein on said “two-dimensional container” namely “substrate” (10), wherein “composition” (6) deposited and fixed in any suitable way as shown in FIG. 1, 2, 6 as one of the possible options.

Such meshwork (8), shown in FIG. 2, including the structure with additional pores (5), as shown in FIGS. 2, 4, additionally ensures reliable retention of “composition”, comprising of particles and molecules hemostatic agents, and contact of latter with the blood, such as devices (11) (12) (13) (14), shown in FIG. 1, 3, 4, and high water absorption and water permeability of “device” further ensuring acceleration of clotting in contact with the blood that flows from the wound.

NAPKIN. In FIG. 6 shown one of the variants of “device”—device (16), consisting of “composition” (6) and “two-dimensional substrate” (10), and preferably, but not limited to square or rectangular or oval, keeps the said “composition” (6). “Substrate” includes special additional pores (5) and irregular grid cell (4) formed weave (1) of fiber and filamentous structures in knots (2, 3). Device (16) is a cloth (FIG. 6) where through said holes (4), and additional holes (5) and through the system of interlacing (1) and knots (2, 3) liquid (blood) can penetrate including due to capillary forces, and interact with the particles and molecules of hemostatic agents of mentioned “composition” (6), resulting in accelerated blood clotting.

Meshwork (FIG. 2) (8) of “substrate” (10) consists of filaments and/or fibers and/or strips that can be interconnected in any suitable way. Interlocking (1) and knots (2.3) between components of mesh structure (8) “substrate” (33) allow material to be flexible and keep constant the size of the holes (4, 5) between them (1,2,3). Yarns, fibers and strips of mesh material can be made of polymers (nylon, polyethylene, polypropylene, polyester, etc.) and/or glass fibers and/or organic matter (natural origin) (e.g., cotton, wool, silk, etc.) and metals and/or their combinations.

For application of device (16) to the bleeding wound, it should be extracted from sealed package and placed over of and/or inside the wound. The particles and molecules of hemostatic agents (6) located in the capacity of the “substrate” (10) contacting with the wound tissue and/or blood, liquid phase is adsorbed on the surface and by the volume of device (16), promoting blood clotting. Flexibility of “substrate” (10) allows a device (16) to take and maintain shape of the wound.

The sealed package (not shown) provides a sterile hemostatic agent prior to its use.

BANDAGE CORRUGATED (90o). Another embodiment of the invention provides that a “device” that as one possible options of hemostatic device—device (21), shown in FIG. 8, consists of two-dimensional hemostatic agent (16), preferably bandage in the form of strip, repeatedly drawn across longitudinal axis (22) on the original tape axis (300), which, as one of the possible examples shown in FIG. 10 forming zigzag folds (23) on the surface of the bandage, whose presence increases the surface area of the hemostatic device contacting surface of damaged tissue (wound), in its turn accelerates blood clotting. The sealed package (not shown) provides a sterile hemostatic agent prior to its use.

For application of device (21) to the bleeding wound, it should be extracted from sealed package and placed over of and/or inside the wound. Mesh material of “substrate” (10) has openings (4, 5), permeable to liquid (blood). The particles and molecules hemostatic agents (6) located in the “substrate” (16) contacting with the wound tissue and/or blood, liquid phase is adsorbed on the surface and by the volume of device (16), promoting blood clotting. Flexibility of material of “substrate” (10) allows it to take and maintain shape of the wound.

BANDAGE CORRUGATED (45°). Another embodiment of the invention provides that “device” that as one possible options of hemostatic device—device (24), is shown in FIG. 10 and comprising two-dimensional hemostatic device (16), preferably bandage in the form of strip, successively or alternative corrugated with angle, preferably but not limited to 45° (as e.g. shown axis (400) in FIG. 10 and device (24) in FIG. 9) relative to the longitudinal axis (22) original strip of bandage sequentially and/or in opposite directions and their combination to form many folds (25) on the surface of the bandage, whose presence increases the surface area of the hemostatic agent in contact with the surface of damaged tissue (wounds), which in turn accelerates blood clotting. The sealed package (not shown) provides a sterile hemostatic agent prior to its use.

For application device (24) to the bleeding wound, it should be extracted from sealed package and placed over of and/or inside the wound. Mesh material of “carrier” (10) has openings (4, 5), permeable to liquid (blood). The particles and molecules hemostatic agents (6) located in the “carrier” (24) contacting with the wound tissue and/or blood, liquid phase is adsorbed on the surface and by the volume of device (24), promoting blood clotting. Flexibility of material of “substrate” (10) allows it to take and maintain shape of the wound.

BANDAGE (ROLL). Device (26) as one possible variants of hemostatic device shown in FIG. 12. This device is a bandage made of “napkin” (16) in the form of strip rolled into a roll (27) for easy application to the wound and bandaging it. For application of device (26) to the bleeding wound, it should be extracted from sealed package and placed over of and/or inside the wound. Mesh material of “substrate” (10) of said bandage (26) has openings (4, 5), permeable to liquid (blood). The particles and molecules hemostatic agents (6) located in the “substrate” (26) contacting with the wound tissue and/or blood, liquid phase is adsorbed on the surface and by the volume of device (26), promoting blood clotting. Flexibility of material of “substrate” (10) allows it to take and maintain shape of the wound. Sufficient length of ribbon collapsed in a roll, can reliably fix the bandage on the wound. If necessary, the bandage can be cut with scissors.

FILM. Another embodiment of the invention provides “device” which, as one possible variant of hemostatic device—device (28), is presented in FIG. 11, and which is a water permeable film (29) having pores (30), permeable for liquid (blood) and may have/or have not additional holes (31) of any shape, preferably but not limited to oval, square, rhombic that is pressed against the surface of the wound and/or placed inside the wound opening. The composition of hemostatic agents (6) incorporated in a water permeable material (29) of device (28).

The material of water permeable film (29) as one of the possible choices can be made, e.g. of polyvinyl alcohol or gelatin, calcium alginate or more.

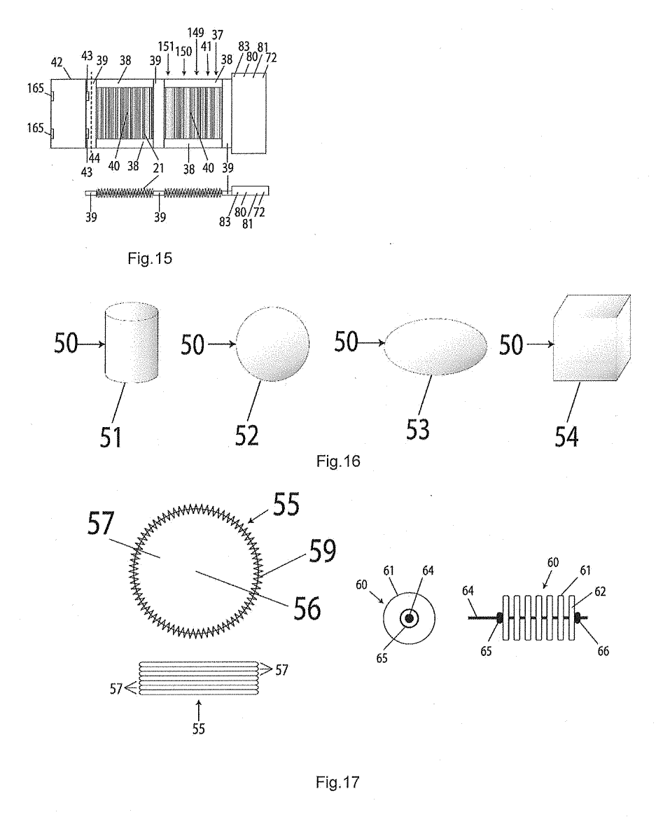

WINDOW-FORM BANDAGE. In FIG. 15 presents one possible variant of hemostatic agent (37) having window-form structure comprising elastic bandage in the form of strip to stop the bleeding, having properties of compressing dressing directly over the wound. This device comprising two ribbons of elastic bandage (38) located on the edges along the longitudinal axis of ribbon and interconnected in any suitable way across the said axis non-elastic strips (39) preferably but not limited to rectangular form in distance preferably but not limited to about 200 mm, thus formed window-form structure of said hemostatic agent. In each “window” (40) inserted ribbon of corrugated bandage (21), which edges in any suitable way fixed to two adjacent non-elastic strips (39), forming “window” (40) with fragments of elastic bandage ribbon (38). Longitudinal and transverse strips made of “two-dimensional” material mainly, but not exclusively, textiles.

Longitudinal strips of device (37) of elastic material are used to create the effect of compressing dressing directly over the wound and secure device on the wound. The transverse strips (39) serve to connect the “windows” (40) with each other, and to preserve the stability of the corrugated surface, which may be aligned with a strong extension of elastic strips (38) along the longitudinal axis of the strip. As the longitudinal strips (38) can be used pieces of elastic bandage in the form of strips, as the cross bars (39) can be used inelastic textile fragments preferably rectangular.

For application of device (37) to the bleeding wound, it should be extracted from sealed package and placed over of and/or inside the wound. The particles and molecules hemostatic agents (6) located in the “substrate” (10) contacting with the wound tissue and/or blood, liquid phase is adsorbed on the surface and by the volume of device (37), promoting blood clotting. Flexibility of “substrate” (10) allows a device (37) to take and maintain shape of the wound. The elasticity of longitudinal strips (38) allows this hemostatic agent to act as compressing dressing directly over the wound. Sufficient length of ribbon preferably collapsed in a roll (26), and the presence of at least one additional special holder (special fasteners) (43) can reliably fix device (37) on the wound. If necessary, the bandage can be cut with scissors.

WINDOW-FORM BANDAGE WITH ATTACHED REGULAR (ELASTIC) BANDAGE. Another embodiment of the invention provides that a “device” that as one possible options of hemostatic device—device (41), shown in FIG. 15 and comprises device (37) as a complex ribbon to one end of which a is its integral part—non-elastic strips (39), connected in any suitable way in the area marked by the dotted line (44) at one end elastic bandage or inelastic bandage (42) to a second end which can be connected special holder (special fastener) (165). Sufficient length of bandage tape (42), and a special clasp (165) additionally provide an opportunity of secure fixation of device (41) on the wound.

Three-dimensional “container” and three-dimensional “substrate” as used herein, means any container having three-dimensional geometric shape preferably but not limited to cylinder, sphere, ellipsoid, a box e.g. in the form of discs, beads, pockets, pouches, pillows, tubes made at least of a fragment of a two-dimensional substrate or device, by fixing at least two opposite edges of the two-dimensional substrate any suitable way and sprays, syringes, tubes, containers and other vessels, including sealed, mainly filled with solid, liquid, semi-liquid, gel-like and paste-like and foam-like spumy and other suitable forms of composition of hemostatic agents, which can also include additional agents having three-dimensional geometric shape preferably but not limited to cylinder, sphere, ellipsoid, a box.

Using three-dimensional hemostatic devices, including tampons, particularly increases sorption capacity of hemostatic device and, consequently, even more to accelerates blood clotting. Increase of sorption capacity of device, for example, can be achieved by combining with each other in any suitable way two and/or more similar and/or different fragments of hemostatic devices, including napkins, bandages, of specific geometry, thus formed “three-dimensional” specific figure, depending on the type of fixation (connection) and/or source fragments and/or the method of fixing the initial fragments. As possible variants of such device (50) on (FIG. 16) made of at least one or two of the same and/or different fragments of hemostatic devices, including wipes, bandage, certain geometric shapes, interconnected by any suitable way, forming of this “three-dimensional” figure of special form preferably, but not limited to cylindrical (51), spherical (52) ellipsoid (53), cubic (54) form. Hemostatic device is pressed to the surface of the wound and/or placed inside the wound opening. Serves for plugging and closing bleeding from surface wounds.

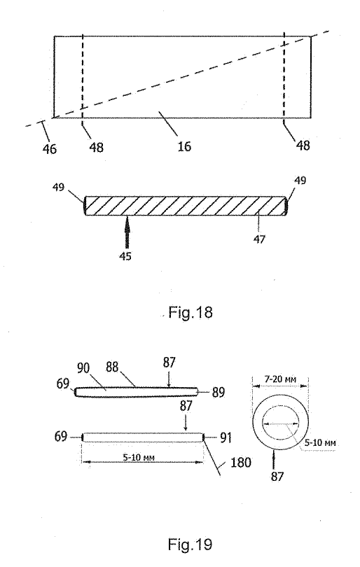

TAMPON “CORD”. Another embodiment of the invention provides that a “device” that as one possible options of hemostatic device—device (45) shown in FIG. 18, wherein it has a cylindrical shape and obtained from the “two-dimensional” rectangular or square hemostatic agent “napkin” type (16) (FIG. 6) of rectangular or square shape by spiral twisting it diagonally (46) to the diameter preferably but not limited to about 5 mm by spiral twisting (47) (clockwise or counterclockwise), has a length preferably, but not limited to about 200-300 mm, which is formed by cutting off unnecessary fragments from both ends and edges by dotted line (48), made by cutting off unnecessary fragments from both ends and edges as formed preferably but not limited to conical or oval shape (49), and attached by any suitable way, including gluing, stitching and others. Device (45) can be used for plugging narrow channels of gunshot wounds and bleeding from the ear canal due to concussion.

TAMPON (DISC AND CYLINDER). As one possible example of such a device (50)-device (55) shown in FIG. 17, and which mostly cylindrical form (51) or disc (200) (not shown) and comprises preferably but not limited to 4-8 layers of fragments (57) of “two-dimensional” hemostatic device (16) in the form of circle fastened (fixed) to each other. In a device of cylindrical shape (51) pieces (57) fastened in any way, particularly stitched in the middle (56), and in device (55) in the form of disk (200) pieces (57) tightly fastened in any way around the perimeter (59). Hemostatic device is pressed to the surface of the wound and/or placed inside the wound opening. Serves for plugging and closing bleeding from surface wounds.

TAMPON “GREMILLE”. Another embodiment of the invention provides that such a device as one of the possible options on hemostatic device—device (60), is shown in FIG. 17, comprising tampon (61) in the form of a cylindrical column (51) with diameter preferably but not limited to about 5-15 mm and length preferably but not limited to about 30-100 mm, formed as a result of a string of discs (62) at their center (56) obtained of hemostatic devices, particularly “napkin” (16), bandage (26), flexible rod (64) with diameter preferably but not limited to 2 mm and a length preferably but not limited to 200 mm, and where tightly compressed on the rod (64) circles (62) in the (61) securely fixed on both sides of his retainers (65), (66) which impaled on the rod (64).

The end of the device (60) is gradually introduced into the wound channel of the retainer (66) by pressing the end of the tampon (61) to opening of said wound channel. Flexibility of rod (64) allows device (60) to deliver hemostatic agents are located in device (60), into the wound opening directly without further injury of the damaged tissue.

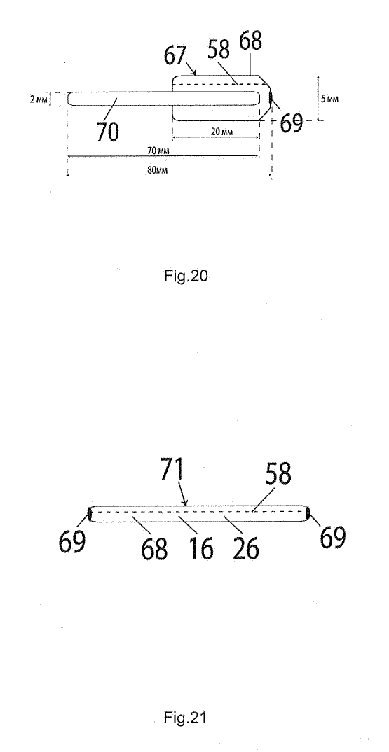

TAMPON ON A FLEXIBLE ROD. Another embodiment of the invention provides that such a device as one of the possible variants on hemostatic device—device (67) shown in FIG. 20 has cylindrical shape (51) is made of a single piece of “two-dimensional” hemostatic devices, including napkins (16), bandage (26) in the form of strip and wherein the said strip material rolled into a roll (68), the free end of which as shown by dotted line (58) parallel to longitudinal axis of is attached by any suitable way to the previous layer to prevent its unwinding and wherein the end (69) of the roll (68) formed by any suitable method for fixing the edge of strip and providing this conical form to the end (69) of the roll (68) to facilitate its entering into the wound and wherein the inner edge of the strip (not shown) additionally fixed by any suitable method at one end of a flexible substrate in the form of rod (70) diameter is preferably, but not limited to about 2 mm, and length preferably, but not limited to about 70 mm and wherein the total length of the said product preferably, but not limited to about 80 mm and wherein the twisted and fixed said “two-dimensional carrier” is preferably, but not limited to about 20 mm in length and diameter preferably, but not limited to about 5 mm. Purpose and application of device (67) are similar to the purpose and application of device (60).

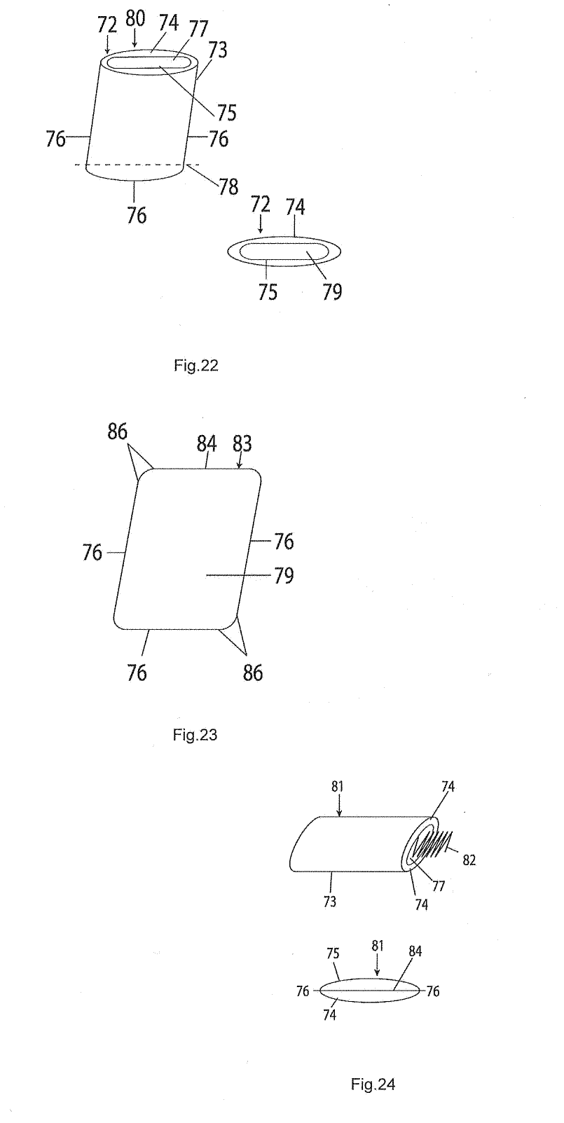

TAMPON “POCKET”. Another embodiment of the invention provides another option of hemostatic devices—device (72) shown in FIG. 22 is made mainly in the form of a pocket (73), presented by container preferably of square or rectangular shape, made by any suitable manner of at least one layer, preferably but not limited to 4-8 layers of “two-dimensional” hemostatic device of bandage (26), or “napkin” (16), type, and where two walls (74, 75) of capacity connected (attached) on three sides (76) by perimeter in any suitable way, and at the fourth side it has a free opening (77) as shown by dotted line on cross section (78) in the middle has a cavity (79). The said device (72) is suitable for plugging wounds by itself and in combination with other agents.

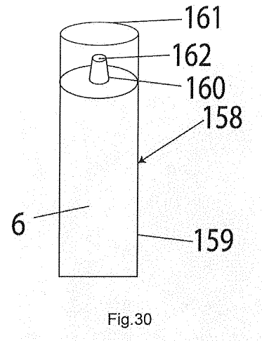

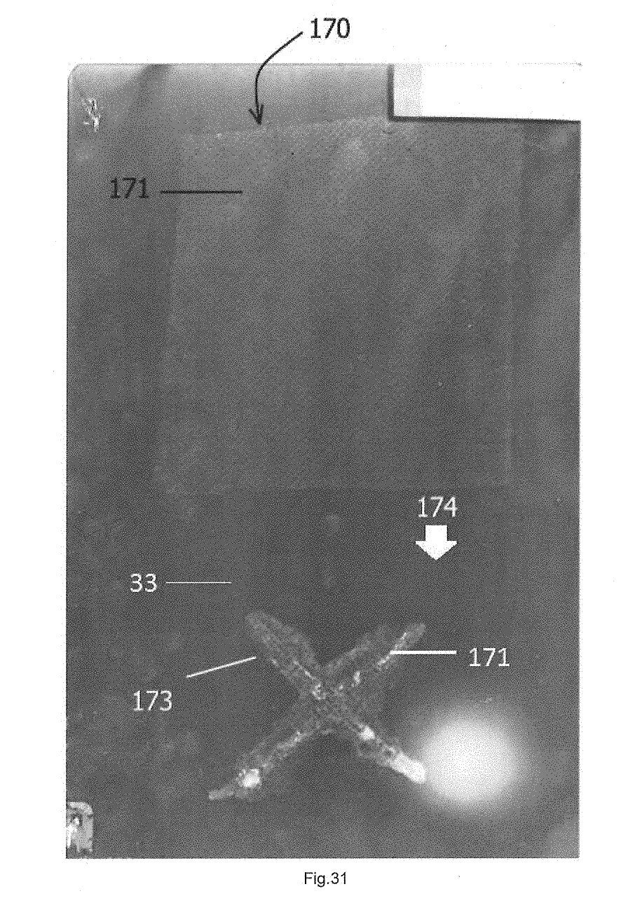

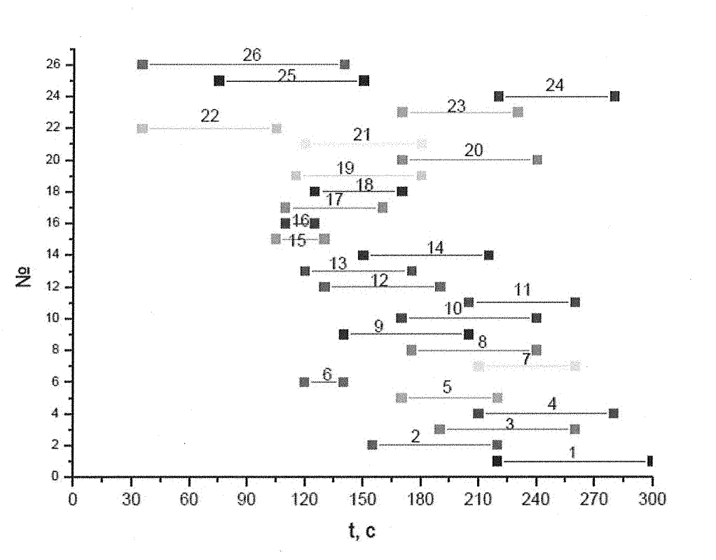

TUBE. In FIG. 30 shown one of possible variants of hemostatic device, characterized in that capacity is vial containing the composition of hemostatic agents in the form of a gel, foam, suspension, paste and at least part of “capacity” for composition of hemostatic agents has water impermeable shell.

Another embodiment of the invention provides that a “device”, as one possible hemostatic device—device (158), shown in FIG. 30 consists of a tube (159) with a spout (160) and a protective cap (161). In the tube (159) is hemostatic composition (6) in the form of a gel, foam, suspension or paste. For application of device (158) to the bleeding wound remove the cap (161), direct the spout (160) to the damaged tissue or wound, squeeze through the hole (162) and put the required number of hemostatic composition to the wound.

TAMPON “HOLLOW STICK”. Another embodiment of the invention provides that a “device”, is one possible hemostatic device—device (71), is presented in FIG. 21, has a cylindrical shape (50) and is made of one piece of one of the “two-dimensional” hemostatic device, including napkins (16) bandage (26), in the form of tape; and wherein said band material coiled into a roll (68) to a thickness in diameter, preferably, but not limited to about 5 mm and which has a length preferably, but not limited to about 200-300 mm, is formed by cutting off unnecessary pieces at both ends and where the free end of the roll (68), shown by the dotted line (58) parallel to the longitudinal axis of the tampon (68) is attached in by suitable manner to the previous layer to prevent its unwinding; and wherein both edges (69) of obtained formed roll (68) connected in any satisfactory way to fix the strip edge and providing conical form to these ends (69) of the roll to facilitate insertion into the wound opening. Designed for plugging tight holes of bullet wounds.

TAMPON (POCKET FILLED WITH OF HEMOSTATIC AGENTS). Another embodiment of the invention provides that a “device”, as one of the options of hemostatic device—device (80), shown in FIG. 22, is a device (72), in which through the free opening (77) inner cavity (79) by any suitable way filled with “two-dimensional” hemostatic agent, chosen preferably, but not limited to the group consisting of wipes (16), bandage (26) including corrugated and/or fragments of these said devices by any suitable way provided specific geometric shape and/or composition of hemostatic agents in the form of paste or dried slurry and combinations thereof, and/or mixtures of the said devices and composition. As one of possible options of the hemostatic device (80) are at FIG. 24 hemostatic device (81), which is a device (72), in which a free opening (77) is inserted into the cavity “two-dimensional” hemostatic device is corrugated bandage (21) with one fixed end inside the pocket to its bottom (84), and the other (82), peeps outward, enabling the use of the said bandage as additional hemostatic device. Said devices (72), (80), (81) are suitable for plugging wounds by itself and in combination with other for devices, such as device (37) or device (41).

TAMPON “PAD”. Another embodiment of the invention provides another option of hemostatic devices—device (83) shown in FIG. 23, comprising a pocket (72) by capacity preferably of square or rectangular shape, made by any suitable manner of at least one layer, preferably but not limited to 4-8 layers of “two-dimensional” hemostatic device of “napkin” (16) or bandage (26) type, and where two walls (74, 75) of capacity connected (attached) on four sides (76), (84) around the perimeter in any way, or in which through the free opening (77) cavity is pre-filled (79) with “two-dimensional” hemostatic device—“napkin” (16), corrugated bandage (21), bandage (26), tampon (80), or (81) or composition of hemostatic agents (6) and combinations thereof, wherein four sides (76), (84) including opening (77) around the perimeter two walls (74, 75) of capacity connected (fixed) in any way, and in addition to the outer surface of one of the edges (76) or (84) attached to any suitable way one end of elastic bandage (not shown) or conventional bandage in length preferably but not limited to about 5 meters, providing fixation of hemostatic device to the wound and/or imposition of compression bandage.

WINDOW-FORM BANDAGE WITH ATTACHED REGULAR (OR ELASTIC) BANDAGES AND PAD. Another embodiment of the invention provides that a “device”, as one possible variants of hemostatic device—device (149), shown in FIG. 15. Device (149) composed of device (37) or device (41) to the one of the edges of the device namely non-elastic strip (39) attached by any suitable way special tampon, namely, the “pad” (83), (FIG. 23). Use of the device “pad” (83) in combination with a bandage of window-form structure (device (37) or device (41)) increases the total sorption capacity of device (149) relative to the blood. To its application to the bleeding wound, tampon—pad (83) should be located on or within the wound and fix using bandage (37 or 41), which also has a hemostatic effect and makes it possible to apply a compressive bandage.

WINDOW-FORM BANDAGE WITH ATTACHED REGULAR (ELASTIC) BANDAGE AND POCKET. Another embodiment of the invention provides that a “device”, as one possible hemostatic device—device (150), shown in FIG. 15 device (150) with composed of device (80) and of device (37) or device (41) to one of the edges of which, namely free non-elastic strip (39) in any suitable way connected special namely “pocket” (80). Use of the device “pocket” (80) (FIG. 15) in combination with a bandage of window-form structure (device (37) or device (41)) increases the total sorption capacity of device (149) relative to the blood. To its application to the bleeding wound, “pocket” (80) should be located on or within the wound and fix using bandage (37 or 41), which also has a hemostatic effect and makes it possible to apply a compressive bandage.

WINDOW-FORM BANDAGE WITH ATTACHED REGULAR (ELASTIC) BANDAGE AND POCKET WITH FIXED INSIDE CORRUGATED BANDAGE. Another embodiment of the invention provides that a “device”, as one possible variants of hemostatic device—device (151), shown in FIG. 15. Device (151) composed of device (37) or device (41) wherein to the one of the edges of the device namely non-elastic strip (39) by any suitable way is attached special tampon (81), namely, the “pocket” (72), with corrugated bandage (21), inside, one edge of which (82) peeps out of the hole of pocket (81). Said corrugated bandage if necessary can be used as an additional hemostatic agent for plugging large wound injuries. Use of the device “pocket” (81) in combination with a bandage of window-form structure (37) or (41) increases the total sorption capacity of device relative to the blood. To its application to the bleeding wound, “pocket” (81) (FIG. 15) should be located on or within the wound and fix using bandage (37 or 41), which also has a hemostatic effect.

TAMPON “FILLED STICK”. Another embodiment of the invention provides that a “device”, is one possible hemostatic device—device (87), is shown in FIG. 19, has a cylindrical shape (51) and is made at least of one piece of one of the “two-dimensional” hemostatic device, including “napkin” (16) bandage (26), in the form of strip and/or at least one layer of “two-dimensional” carrier; and wherein said band material coiled into a roll (88) free end of the roll shown by the dotted line parallel to the longitudinal axis of the is attached in by suitable manner to the previous layer to prevent its unwinding; and wherein one end (69) of obtained formed roll (88) connected in any suitable way to fix the strip edge and providing conical form of this roll (69) to facilitate insertion into the wound opening, and wherein through free opening (89) cavity (90) prefilled with “two-dimensional” hemostatic device, chosen preferably but not limited to the group of “napkin” (16) bandage (26), including corrugated (21) and/or fragments of these devices shaped in any suitable way into certain geometry, or composition of hemostatic agents (6) in form of paste or dried suspension and combinations thereof and/or material selected from the group preferably but not limited to cotton, viscose and other, and/or a mix of said device and/or said composition and where the end (69, 91) free opening (89) which cylinder with preferably, but only with one end conical and has preferably but not limited to the following dimensions: about 50-200 mm in length and a of about 7-20 mm in diameter, diameter of inner cavity is about 5-10 mm. For ease of extraction from wound such device (87) can further have applicator (180) in the form of thread, ribbon, strip.

Such device (87) designed for plugging tight holes of bullet wounds, nasal and ear bleeding.

TAMPON. Another embodiment of the invention provides that a “device”, is one possible hemostatic device—device (87), is presented in FIG. 19, has a cylindrical shape (51) and is made at least of one piece of one of the “two-dimensional” hemostatic device, including napkins (16) bandage (26), in the form of strip and/or at least one layer of “two-dimensional” substrate; and wherein said band material coiled into a roll (88) free end of the roll shown by the dotted line parallel to the longitudinal axis of the is attached in by suitable manner to the previous layer to prevent its unwinding; and wherein one end (69) of obtained formed roll (88) connected in any satisfactory way to fix the strip edge and providing conical form of this roll (69) to facilitate insertion into the wound opening, and wherein through free opening (89) cavity (90) prefilled with “two-dimensional” hemostatic agent, chosen preferably but not limited to the group of “napkin” (16) bandage (26), including corrugated (21) and/or fragments of these devices shaped in any suitable way into certain geometry, or composition of hemostatic agents (6) in form of paste or dried suspension and combinations thereof and/or material selected from the group preferably but not limited to cotton, viscose and other, and/or a mix of said device and/or said composition and where the end (91) free opening (89) bears closed in any suitable way to fix the edge strip (91), and where the device (87), which cylinder with preferably, but only with one end conical and has preferably but not limited to the following dimensions: about 40-70 mm in length and a of about 7-20 mm in diameter, diameter of inner cavity is about 5-10 mm. For ease of extraction from wound such device (87) can further have applicator (180) in the form of thread, ribbon, strip.

As one of the possible options of such device (87) may be within introducer (not shown), optionally designed as a syringe of cylindrical shape in which one end of the touches the piston and the other end touching the opposite end of the syringe, comprising at least four petals that capture the in the cylinder, and allowing leave the syringe cylinder and get into the wound. Such device (87) designed for plugging tight holes of bullet wounds, for intravaginal and rectal application.

TAMPON. Another embodiment of the invention provides that a “device”, as one possible variants of hemostatic device—device (87), shown in FIG. 19 has cylindrical shape (51). This roll (88), comprising capacity of cylindrical form (51) can also be rolled up of at least one layer of “two-dimensional” hemostatic device and/or of at least one layer of “two-dimensional” hemostatic device. Formed cylindrical form corresponds the shape of the inner formed of at least one piece of “two-dimensional” hemostatic device and/or rolled and/or twisted and/or stranded and/or compressed of several pieces of “two-dimensional” hemostatic device and fixed in suitable way. Said inner swab, connected with container in any suitable way preferably, but not limited to stitched to container along longitudinal axis of cylinder from one edge to other and wherein inner tampon additionally compressed and wherein free ends of layer of “two-dimensional” substrate and/or layer of “two-dimensional” hemostatic device are parallel to the longitudinal axis of cylinder freely sliding along the surface of inner swab, that further providing the ability to several fold volume increase of compressed after contact with blood in the wound, and wherein the tampon has preferably, but not limited to the following dimensions: about 40-70 mm in length and about 10-15 mm in diameter. As one of the possible options of such device (87) may be within introducer (not shown), optionally designed as a syringe of cylindrical shape in which one end of the tampon touches the piston and the other end touching the opposite end of the syringe, comprising at least four petals that capture the tampon in the cylinder, and allowing tampon leave the syringe cylinder and get into the wound, and an additional applicator (not shown) preferably strips or yarn or ribbon attached to the tampon (88), provides ease of removal of tampon from the wound. Such device (87) designed first of all for intravaginal and rectal application.

TAMPON. Another embodiment of the invention provides that a “device”, as one possible variants of hemostatic device—device (87), shown in FIG. 19. This roll (88) has a cylindrical shape (51), wherein two ends of the cylinder with the conical bottom, and wherein cylinder is preferably but not limited to the following dimensions: length of about 50-200 mm and diameter of about 7-20 mm, diameter inner cavity 5-10 mm. Such device (87) designed for plugging narrow channels of bullet wounds, nasal and ear bleeding.

TAMPON WITH INTRODUCER. As one of the possible options of such device (87) shown in FIG. 19, may be within introducer (not shown), optionally designed as a syringe of cylindrical shape in which one end of the tampon touches the piston and the other end touching the opposite end of the syringe, comprising at least four petals that capture the in the cylinder, and allowing tampon leave the syringe cylinder and get into the wound, wherein said preferably but not limited to following dimensions: length about 50-70 mm and diameter about 10-20 mm, securing ease of introducing tampon (88) into the wound and an additional applicator (not shown) preferably strips or yarn or ribbon attached to the tampon (88), provides ease of removal of tampon from the wound. Such device designed first of all for intravaginal and rectal application.

Device (87) designed for plugging narrow channels of bullet wounds, nasal and ear bleeding as well for intravaginal and rectal application.

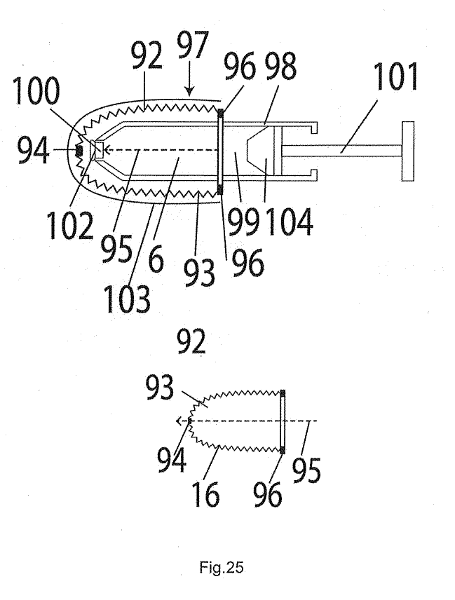

SAC CORRUGATED. Another embodiment of the invention provides that a “device”, as one possible variants of hemostatic device—device (92), shown in FIG. 25. This device comprising sac (93) made of “two-dimensional” hemostatic device, particularly “napkin” (16), and wherein the two edges of the mentioned device interconnected of cylindrical shape in any satisfactory way. On the one side of base of cylinder the edges connected (94) by any suitable way and form cone bottom shape or round shape, Corrugated cylinder walls formed mainly, but not limited to in parallel or perpendicular to its longitudinal axis (95). On the other side to cylinder around the perimeter of its base can be attached waterproof rubber ring (96), which under certain conditions can close the (93) as a diaphragm. Device (92) can be further filled with composition of hemostatic agents or hemostatic devices.

SYRINGE WITH SAC CORRUGATED. Another embodiment of the invention provides that a “device”, as one possible variants of hemostatic device—device (97), shown in FIG. 25. Device (97) consists of “device” (92) and a special device—introducer in the form of a syringe (98) is filled with composition of hemostatic agents (6) in form of gel (99) and/or suspension (99) and/or paste (99) and/or a combination of the above (99). On one hand syringe (98), corked with stopper (100), tightly closing special opening (102), holds hemostatic agents (6, 99) inside the syringe (98), but can be easily removed by pressing the plunger (101) of piston (104). Piston (104) has similar to a conical shape corresponding to conical shape of the inner surface of the syringe (98) at its outlet and provides quantitative squeezing of composition of hemostatic agents (6, 99) through the opening (102).

With free outside of the syringe (98) is set device (92) is made in the form of an empty sac (93) and ring (96) which when clicked rod (101) of the piston (104) moves freely on the outer surface of the syringe (98) and that after the subsidence of the outer surface of the syringe (98) covers the full with composition of hemostatic agents (6) in form of gel (99) and/or suspension (99) and/or paste (99) and/or a combination of the above (99) sac (93), a diaphragm (not shown) and remains outside the wound. From the outside the bag closed with cap (103) to prevent its deformation during packaging and storage. For application of device (97) to the wound bleeding need to extract hemostatic device from the sealed package, remove the cap (103), press or close with distal from the piston end (94) to the wound, gradually introduce to the optimum depth of the wound hole and press on rod (101) of the piston (104) until it stops, so that the composition of hemostatic agents (6, 99) squeezed from a syringe (98) and filling the sac (93) directly in the wound, so that the sac (93) of device (97) placed in the wound and fully meets its shape and size.

Another embodiment of the invention provides that a “device”, as one possible variants of hemostatic device—device (97), shown in FIG. 25. Device (97) wherein the sac (93) closed with cap (not shown) made in the form of cylinder, with bottom comprising at least four petals diverging under pressure of sac (93) shifted from the introducer case (98) the extent of filling with solution, suspension or gel of hemostatic compositions (6) upon pressure on the rod (101) of the piston (104), which provides sterile plugging the wound and where to avoid deformation of cap petals impaled on him an additional protective cap (103) to be removed immediately before application to the wound.

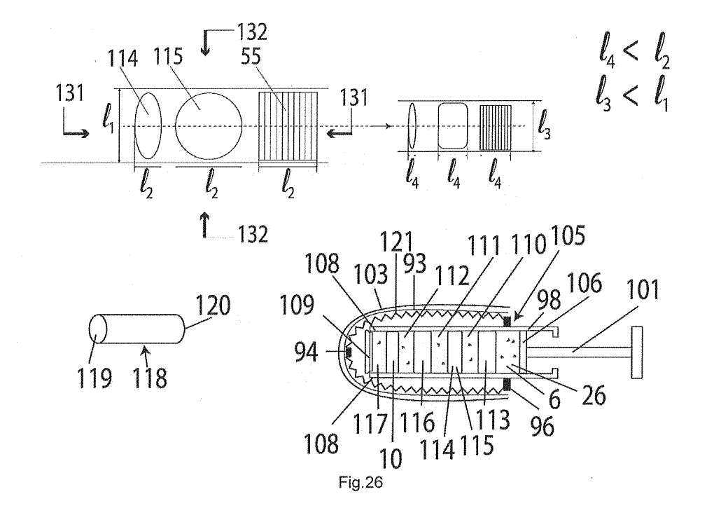

SYRINGE FILLED WITH DRY HEMOSTATIC DEVICES AND/OR COMPRESSED TAMPONS AND HEMOSTATIC COMPOSITION. Another embodiment of the invention provides that a “device”, as one possible variants of hemostatic device—device (105), shown in FIG. 26. Device (105) comprises device (92) and device-introducer in the form of syringe (98) filled with “two-dimensional” hemostatic device (107). The above device (107), selected preferably but not limited to group comprising “napkin” (16) bandage (26) of corrugated bandage (21) and/or “two-dimensional” “substrate” (10), and that any suitable way provided certain geometry (110), and/or composition of hemostatic agents (6) as a paste or dried suspension, and combinations thereof (111), and/or individual hemostatic agent and/or a mixture of the above devices, “substrate”, of hemostatic agents and/or hemostatic composition (112) and/or produced (113) with a “two-dimensional” hemostatic device preferably tampons ((55) cylinder), sponges ((114) disk), spheres (115)), which at least one axis form a three-dimensional shape, which in cross section along this axis is a circle (cylinders, discs, spheres), and that the maximum compressed along the axis and/or perpendicular to it direction, ensuring minimization of volume compared to their up to compression and/or a mixture of the above devices and said composition (117). The above said components ((110), (111), (112), (113), (114), (115), (117)), in any order and in any way placed in a syringe inane, and upon compression in the axis along which moves a piston to form additional hemostatic device (118) tightly compressed in a syringe inane, so that is convenient for getting a wound cylindrical shape. On the one hand syringe (98) plugged via plug (109) which holds the hemostatic device (118) within the syringe (98), but can be easily removed through a hole (108) is in form and size corresponds to the diameter of the edge (119) of the compacted device (118), and wherein the piston (106) has a cylindrical shape, ensuring comfort and completeness squeezing mentioned means (118), the action of one party (120) whose (118) piston (106) when you press the rod (101). The outer surface of the syringe (98) from the free hole (108) strained device (92) is made in the form of an empty (93), the folds of which (121) are focused mainly on the edge (108) of the syringe (98) and a ring which (96) freely moving on the outer surface of the syringe (98) and remains outside the wound. From the outside the sac closed with cap (103). For application device (105) to the wound bleeding need to extract hemostatic device from the sealed package (not shown), remove the cap (103), press the opposite piston end to the wound, and click on the rod (101) of the piston (106) to stop, causing the piston (106) presses the edge (120) of device (118) which presses on the bottom of the sac (93), shifting it into deep wound and fills a sac (93) directly in the wound, so that device (105) which is formed by the filling device (92) with device (118) directly into the wound, is placed in the wound and fully meets its shape and size. Thus, the resulting ring (96) slides on the outer surface of the syringe (98) and after movement of the syringe (98) in the opposite direction heading off the edge of the ring (108) syringe and diaphragm (not shown) is automatically triggered to close hole sac. The ring serves as an additional applicator for easy removal device (105) of the wound.

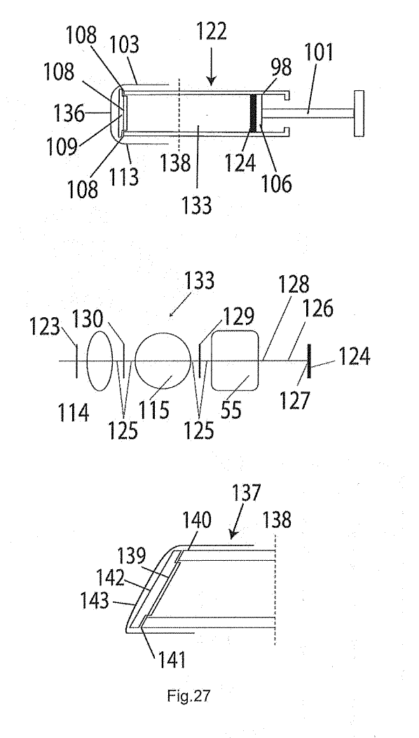

SYRINGE FILLED WITH DRY HEMOSTATIC DEVICES PREFERABLY COMPRESSED AND/OR CONNECTED, SPONGES, ETC. Another embodiment of the invention provides that a “device”, as one possible variants of hemostatic device—device (122), shown in FIG. 27. Device (122) consists of “three-dimensional” hemostatic device (133) and device—introducer syringe (98) filled with “three-dimensional” hemostatic device (133). The said device (133) made at least of two identical or different hemostatic agents, chosen primarily, but not limited to the group, consisting mainly of tampons, sponges made primarily of “two-dimensional” hemostatic device, forming at least by one axis a three-dimensional shape shown in FIG. 26, and in cross section along this axis (116) is a circle, particularly cylinders (55), discs (114), inanes (115), and/or similar three-dimensional devices, that the maximum uncompressed along the (131) the axis and/or perpendicular to it direction (132) and dimensions after compression at least in one direction smaller before compression, particularly parallel (131) and/or perpendicular (132) to the axis (not shown) on which moves a piston, ensuring minimization of volume compared to their volume before compression, one of the options shown in FIG. 27 additionally includes fragments of the appropriate form “two-dimensional” hemostatic devices selected preferably but not limited to the group of “napkin” (16) bandage (26), film (28), wherein said “three-dimensional” devices and/or fragments (129), (130), (123), “two-dimensional” devices (16) (28) and/or “substrate” (10) preferably a circle additionally connected (stitched) together preferably at the geometric center (128) above the circle (not shown) in any order and in any suitable way, preferably but not limited to stitched, preferably with thread, strip (125), preserving the same or different distances between them. The above said interconnected and tightly compressed components are located in the inane syringe form a hemostatic agent cylindrical device (133), which is convenient for getting into the wound cylindrical shape, and one end (127) most extreme thread or tape (126) fixed to washer (124) which is attached to the end (not shown) of the piston (106), the syringe (98), and that the reverse course of the piston (106) of the syringe (98) is separated from the piston remains above the wound and serves as an applicator that provides ease of removal of medium from the wound. For convenience and completeness of squeezing said hemostatic device (133) cylindrical in wound size and shape of the hole edge (108) of the syringe (98) corresponds to the diameter of the compressed device (133) and piston (106) has a cylindrical shape.

For application device (122) to the wound bleeding need to extract hemostatic device from the sealed package (not shown), remove protective cap (103), press the opposite piston end to the wound, and click on the rod (101) of the piston (106) to stop, causing the piston (106) presses the edge (122) squeezing device into deep wound due to that device (133) is placed in the wound and fully meets its shape and size. Upon this reverse course of the piston (106) washer (124) separating from it and stay over wound as handy applicator device for extracting device (122) from the wound. Upon getting into the wound and in contacting with blood device (122), it increases in volume in all directions, facilitating effective plugging the wound opening.

SYRINGE WITH A BEVELED END FILLED WITH DRY HEMOSTATIC DEVICES PREFERABLY COMPRESSED AND/OR CONNECTED SWABS, SPONGES, ETC. Another embodiment of the invention provides that a “device”, as one possible variants of hemostatic device—device (137), shown in FIG. 27. Device (137) consists of “three-dimensional” hemostatic device (133) and device—introducer syringe (98) filled with “three-dimensional” hemostatic device (133). The said device (133) made of at least two identical or different hemostatic agents, chosen primarily, but not limited to the group, consisting mainly of tampons, sponges made primarily of “two-dimensional” hemostatic device, forming at least by one axis a three-dimensional shape shown in FIG. 26, and in cross section along this axis (116) is a circle, particularly cylinders (55), discs (114), spheres (115), and/or similar three-dimensional devices, that the maximum compressed along the (131) the axis and/or perpendicular to it direction (132) and dimensions after compression at least in one direction smaller before compression, particularly parallel (131) and/or perpendicular (132) to the axis (not shown) on which moves a piston, ensuring minimization of volume compared to their volume before compression, one of the options shown in FIG. 27 additionally includes fragments of the appropriate form “two-dimensional” hemostatic devices selected preferably but not limited to the group of “napkin” (16) bandage (26), film (28), wherein said “three-dimensional” devices and/or fragments (129), (130), (123), “two-dimensional” devices (16) (28) and/or “substrate” (10) preferably a circle additionally connected (stitched) together preferably at the geometric center (128) above the circle (not shown) in any order and in any suitable way, preferably but not limited to stitched, preferably with thread, strip (125), preserving the same or different distances between them. The above said interconnected and tightly compressed components are located in the inane syringe form a hemostatic agent cylindrical device (133), which is convenient for getting into the wound cylindrical shape, and one end (127) most extreme thread or tape (126) fixed to washer (124) which is attached to the end (not shown) of the piston (106), the syringe (98), and that the reverse course of the piston (106) of the syringe (98) is separated from the piston remains above the wound and serves as an applicator that provides ease of removal of medium from the wound. Said syringe has any suitable form preferably, but not limited to the cylinder. Unlike hemostatic device (133) said hemostatic device (137) as possible options of device shown in FIG. 27 shall end tapered shape, and as shown in the cross opening device along the axis (138), which is shown by the dotted line, its upper end (140) is longer than the bottom (141), and its hole (139) is closed stopper (142), the amount which satisfactorily meet the size and shape of the hole (139) and which closed cap (103) or cap (143). This tapered shape syringe device (137) further provides the convenience of fixing it in the wound and entering the wound and therefore provides an opportunity to enter hemostatic agent (133) in the wound opening. For convenience and completeness of squeezing said hemostatic device (133) cylindrical in wound size and shape of the hole edge (139) of the syringe (98) corresponds to the diameter of the compressed device (133) and piston (106) has corresponding cylindrical shape.

For application device (137) to the wound bleeding need to extract hemostatic device from the sealed package (not shown), remove the cap (103) (or (143)), sharp edge beveled end (141) a little put it to the wound and press on the rod (101) of the piston (106) to stop, causing the piston (106) presses the edge device (133) and squeezing means (133) deep into the wound, making device (133) placed in the wound and fully meets its shape and size. Upon getting into the wound and in contacting with blood device (133), it increases in volume in all directions, facilitating effective plugging the wound opening.

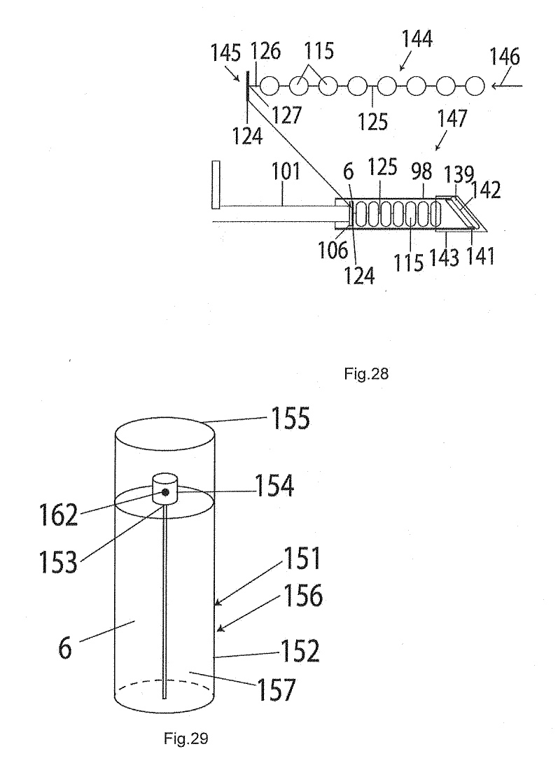

TAMPON “BEADS” INTERCONNECTED. For plugging of deep wound holes can also be used device (144) (FIG. 28), comprising tampon of interconnected beads (115). Spheres (115) can be obtained of “two-dimensional” hemostatic device in particular, napkins (16), film (28) bandage (21), (26) and/or its fragments in any suitable way or of “two-dimensional” substrate in particular fragments of textiles—woven and/or nonwoven, and/or fibrous materials, including cotton, viscose, wool etc., formed in the shape of spheres (115) in every suitable way, followed by connection of fragments of said device in the form of spheres (115) one by one together using threads and/or tapes in any suitable way. The shape and size of the spheres (115) ensuring their optimal location in the wound opening. Spheres can be further compressed (54), (115) as shown in FIG. 16, 26. With thread and/or strip (125), connecting beads (115), or with end (127) extreme thread (126) or strip, disposed tampon can be extracted from the wound opening. Beads can also be further compressed at least two sides in at least one of the areas—mainly parallel (134) and/or perpendicular (135) to the axis (146) (FIG. 28) where beads connected with threads and/or strips. If compression beads occupy less volume and upon contacting with blood in the wound significantly increases its volume, they are expanding, and this facilitates more efficient plugging the wound.

For plugging of deep wound holes can also be used device (144) (FIG. 28), is a tampon comprising tightly interconnected beads (115) of hemostatic device in diameter preferably but not limited to 5 mm to 20 mm preferably but not limited to the same diameter, using threads and/or tapes (125) at a distance preferably but not limited to about 5-50 mm. Thread and/or ribbon (125) that connects them made of any suitable material. Balls can be obtained of “two-dimensional” hemostatic device in particular, “napkin” (16), film (28) bandage (21), (26) and/or its fragments in any suitable way or of “two-dimensional” substrate in particular fragments of textiles—woven and/or nonwoven, and/or fibrous materials, including cotton, viscose, wool etc., formed in the shape of spheres (115) in every suitable way, followed by connection of fragments of said device in the form of spheres (115) one by one together using threads and/or tapes in any suitable way. The shape and size of the spheres (115) ensuring their optimal location in the wound opening. Spheres can be further compressed (54), (115) as shown in FIG. 16, 26. With thread and/or strip (125), connecting spheres (115), or with end (127) extreme thread (126) or strip, disposed can be extracted from the wound opening. Spheres can also be further compressed at least two sides in at least one of the areas—mainly parallel (134) and/or perpendicular (135) to the axis (146) (FIG. 28) where spheres connected with threads and/or strips. If compression spheres occupy less volume and upon contacting with blood in the wound significantly increases its volume, they are expanding, and this facilitates more efficient plugging the wound.

For application device (144) to the bleeding wound, it should be extracted from sealed package (not shown) and placed over of and/or inside the wound by the gradual introduction of interconnected spheres (115) one after another. The latter thread and/or rope (126) connecting said spheres and/or end (127) of latter thread or strip (or the latter before the end (127) of sphere or spheres) remains outside the wound hole and serves for the removal of the tampon from wound. Flexibility of material allows device (144) to take and maintain shape of the wound. Device (144) is designed for plugging deep wound surfaces preferably, but not limited to with a narrow inlet.

“BEADS” INTERCONNECTED WITH ATTACHED WASHER. For easier removal tampon from the wound may be used device (145), shown in FIG. 28 and differs from (144) in that it has an additional applicator (124), preferably but not limited to of rubber washer (124), attached to the free end (127) of thread or strip (126). Application of device (145) are similar to application of device (144). Pull the washer (124) to remove the tampon from the wound.

SYRINGE FILLED WITH “BEADS” ATTACHED WASHER. Another embodiment of the invention provides that a “device”, as one possible variants of hemostatic device—device (147), shown in FIG. 28. Device (147) comprising tightly packed device (145) placed inside the syringe type device-introducer (98) preferably having form of truncated at acute angle cylinder (139). Such form facilitates easy directed application of device (145) into the wound channel. Said part of the syringe (139) closed with cap (143), whose shape and size corresponding to the shape and size of beveled edge (139).

Spheres (115) of device (145), tightly packed (148) into the preferably cylindrical syringe (98) and firmly connected to the piston (106) by thread (126), attached to its end (127) to rubber washer (124) fixed to the inner side of the piston (106). The rubber washer (124) capable to by easily disconnected from the piston (106) during reverse the latter. Spheres (115) of device (145) can be further compressed.

For application of device (147) to the wound bleeding need to extract device from the sealed package, remove the protective cap (143), remove the stopper (139) and press the end (141) of device to wound hole, put it in an optimal depth and press the rod (101) of the piston (106) then remove the syringe from the wound so to disconnect washer (124) from piston (106) and remain out of the wound. The particles and molecules of hemostatic composition (6) located in the spheres of “carrier” (148) of device (145) contacting with the wound tissue and/or blood, liquid phase is adsorbed on the surface and by the volume of spheres, promoting blood clotting. This tightly packed and/or compressed balls increased in size, which contributes to plugging the wound.

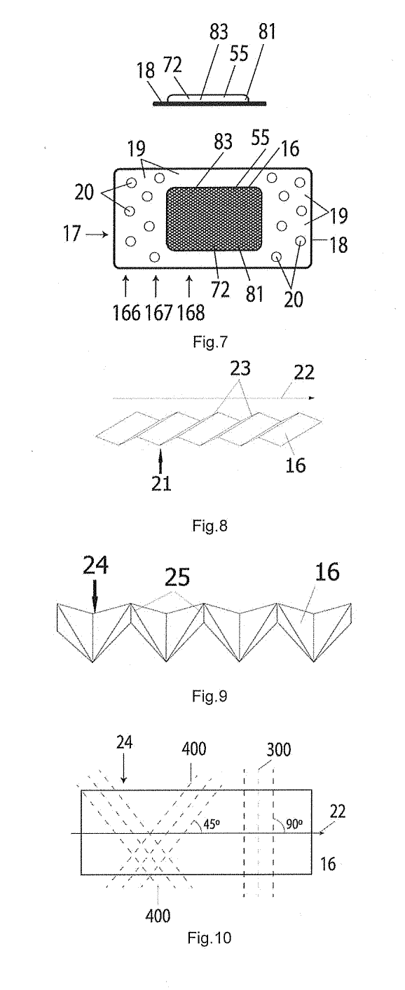

PATCH. In FIG. 7 shown one possible variant of hemostatic device—device (17), “two-dimensional” substrate (33) preferably but not limited to rectangular form, at least part of which impregnated with a composition of hemostatic agents (6) and forms a hemostatic device “napkin” (16) preferably but not limited to in the form of a rectangle, square, diamond, circle, and wherein at least part of the surface of said dimensional” substrate (33) at least on one side is covered with a covered with a thin sticky layer of pressure sensitive adhesive (19), securing its adhesion to patient's skin.

Device (17) is located at the patient's wound so that hemostatic material (16) directly contacting with damaged skin surface (wound), and the adhesive surface (19) of the “two-dimensional”substrate (33) fixed (covering surface of the patient's skin) around the wound. “Two-dimensional” substrate (33) includes a hole (20) ensuring evaporation of moisture from the skin.

PATCH. In FIG. 7 shown one possible variant of hemostatic device—device (17), comprising device (16) which is attached to the elastic polymer and/or plastic and/or woven and/or non-woven base (18) preferably, but not limited to rectangular, and wherein part of surface of said elastic base (18) at least on one side covered with adhesive layer of pressure sensitive adhesive agent (19), securing its adhesion to patient's skin.

Device (17) is located at the patient's wound so that hemostatic material device (16) directly contacting with damaged skin surface (wound), and the adhesive surface (19) of the elastic base (18) fixed (covering surface of the patient's skin) around the wound. Elastic base (18) includes a hole (20) ensuring evaporation of moisture from the skin.

PATCH. Another embodiment of the invention provides another option of hemostatic devices—device (166) shown in FIG. 7, comprising device (83), comprising a pocket (73) presented by capacity preferably of square or rectangular shape, made by any suitable manner of at least one layer, preferably but not limited to 4-8 layers of “two-dimensional” hemostatic device of “napkin” (16) or bandage (26) type, and where two walls (74, 75) of capacity connected (attached) on four sides (76), (84) around the perimeter in any way, or in which through the free opening (77) cavity is pre-filled (79) with “two-dimensional” hemostatic device —“napkin” (16), corrugated bandage (21), bandage (26), tampon (82), or composition of hemostatic agents (6) and combinations thereof, wherein four sides (76), (84) including opening (77) around the perimeter two walls (74, 75) of capacity connected (fixed) in any way, and wherein device (83) “pad” attached to the elastic polymer and/or plastic and/or woven and/or non-woven base (18) preferably but not limited to rectangular, wherein the surface of said elastic base (18) at least on one side is covered with a covered with a thin sticky layer of pressure sensitive adhesive (19), securing its adhesion to patient's skin.

Device (166) is located at the patient's wound so that hemostatic material (16) directly contacting with damaged skin surface (wound), and the adhesive surface (19) of the elastic base (18) fixed (covering surface of the patient's skin) around the wound. Elastic base (18) includes a hole (20) ensuring evaporation of moisture from the skin. Presence of device (83) in the device (166) provides increased sorption capacity of device (166) and, accordingly, its hemostatic effect.

PATCH. Another embodiment of the invention provides another option of hemostatic devices—device (167) shown in FIG. 7, comprises device (55) and which mostly cylindrical form (51) or disc (200) (not shown) comprises preferably but not limited to 4-8 layers of fragments (57) of “two-dimensional” hemostatic device “napkin” (16) are fastened (fixed) to each other in any suitable way around the perimeter (56) and/or stitched in the middle (56), and where the device (55), “DISC” (FIG. 17), attached (FIG. 7) to the elastic polymer and/or plastic and/or woven and/or non-woven base (18) preferably but not limited to rectangular, and wherein part of surface of said elastic base (18) at least on one side covered with thin sticky layer of pressure sensitive adhesive agent (19), securing its adhesion to patient's skin.

Device (167) is located at the patient's wound so that hemostatic material (16) directly contacting with damaged skin surface (wound), and the adhesive surface (19) of the elastic base (18) fixed (covering surface of the patient's skin) around the wound. Elastic base (18) includes a hole (20) ensuring evaporation of moisture from the skin. Presence of device (55) in the device (167) provides increased sorption capacity of device (167) and, accordingly, its hemostatic effect. Hemostatic device is pressed to the surface of the wound. Serves for plugging and closing bleeding from surface of bleeding wounds.

PATCH. Another embodiment of the invention provides another option of hemostatic devices—device (168) shown in FIG. 7, comprising of device (72), shown in FIG. 22 and comprising pocket (73) presented by “container” preferably of square or rectangular shape, made by any suitable manner of at least one layer, preferably but not limited to 4-8 layers of “two-dimensional” hemostatic device of bandage (26) or “napkin” (16), type, and where two walls (74, 75) of capacity connected (attached) on three sides (76) by perimeter in any suitable way, and at the fourth side it has a free opening (77) as shown by dotted line on cross section (78) in the middle has a cavity (79), wherein through free opening (77) inner cavity (79) by any suitable way additionally filled with “two-dimensional” hemostatic device, preferably but not limited to “napkin” (16), bandage (26), particularly corrugated and/or fragments of these devices, by any suitable way provided certain geometry and/or composition of hemostatic agents in the form of paste, dried suspension, film or sponge (not shown) made, for example, of calcium alginate and combinations thereof, and/or mixtures of the above agents and forms of composition wherein device (72) attached to the elastic polymer and/or plastic and/or woven and/or non-woven base (18) preferably but not limited to rectangular wherein part of the surface of said elastic base (18) at least on one side is covered with a covered with a thin sticky layer of pressure sensitive adhesive (19), securing its adhesion to patient's skin.

Device (168) is located at the patient's wound so that hemostatic material (16) directly contacting with damaged skin surface (wound), and the adhesive surface (19) of the elastic base (18) fixed (covering surface of the patient's skin) around the wound. Elastic base (18) includes a hole (20) ensuring evaporation of moisture from the patient's skin. Presence of device (72) in the device (168) provides increased sorption capacity of device (168) and, accordingly, its hemostatic effect. Hemostatic device is pressed to the surface of the wound. Serves for plugging and closing bleeding from surface of bleeding wounds.

PATCH TAMPON “POCKET”+CORRUGATED BANDAGE. Another embodiment of the invention provides another option of hemostatic devices—device (169) shown in FIG. 7, comprising of device (81), shown in FIG. 24 comprising a pocket (73), presented by container preferably of square or rectangular shape, made by any suitable manner of at least one layer, preferably but not limited to 4-8 layers of “two-dimensional” hemostatic device of bandage (26), or “napkin” (16), type, and where two walls (74, 75) of container connected (attached) on three sides (76) by perimeter in any suitable way, and at the fourth side it has a free opening (77) as shown by dotted line on cross section (78) in the middle has a cavity (79), wherein through free opening (77) in the cavity inserted “two-dimensional” hemostatic device—corrugated bandage (21), which one end fixed inside the pocket to its bottom, and the other (82) peeps outward, enabling use of the bandage as additional hemostatic agent, wherein the device (81) attached to the elastic polymer and/or plastic and/or woven and/or non-woven base (18) preferably but not limited to rectangular, wherein the surface of said elastic base (18) at least on one side is covered with a covered with a thin sticky layer of pressure sensitive adhesive (19), securing its adhesion to patient's skin.

Device (169) is located at the patient's wound so that hemostatic material (16) directly contacting with damaged skin surface (wound), and the adhesive surface (19) of the elastic base (18) fixed (covering surface of the patient's skin) around the wound. Elastic base (18) includes a hole (20) ensuring evaporation of moisture from the patient's skin. Presence of device (81) in the device (169) provides additional opportunities of plugging wounds and increased sorption capacity of device (169) and, accordingly, its hemostatic effect. Hemostatic device is pressed to the surface of the wound. Serves for plugging and closing bleeding from surface of bleeding wounds.

TUBE. As possible options of hemostatic agent in FIG. 30 shown device (158)—vessel (tube) (159)—“container”, mostly is waterproof membrane, comprising composition (6) of hemostatic agents, comprising gel, foam, paste, slurry, and one specific variant of foam shown in the example No 27, one specific variant of gel shown in the example No 10, or one specific variant of suspension shown in the example 12, or one specific variant of paste shown in the example No 6.

Device (158), shown in FIG. 30 has tube (159) with a spout (160) with a hole (162) and a protective cap (161). In the tube 159 is hemostatic composition (6) in the form of a gel, foam, suspension or paste. For application of device (158) to the bleeding wound remove the cap (161), direct opening (162) of the spout (160) to the damaged tissue or wound, squeeze and put the required number of hemostatic composition to the wound.

Another embodiment of the invention provides that a “device”, as one possible hemostatic device—device (151) in which part of the “capacity” has a waterproof shell, shown in FIG. 29 consists of a balloon (152) valve (153) atomizer (154) and a cap (155) protecting atomizer.

SPRAY FOAM. Another embodiment of the invention provides that a “device”, as one possible hemostatic device—device (156), presented in FIG. 29 consists of a balloon (152) valve (153) atomizer (154) and a cap (155) protecting atomizer. In the container (152) is a hemostatic composition given as one of the options in the example No 27. By pressing the valve (153) of sprayer (154) this composition due to the presence in it water-retaining hemostatic agent carboxymethyl cellulose, which is a foaming agent, and additional foaming agent (157), which is an organic hemostatic agent polyvinyl acetate, which is part of said hemostatic composition goes through the hole (162) as a foam. For application of device (156) to the bleeding wound remove the cap (155), direct opening (162) of the atomizer (154) to the damaged tissue or wound, push valve (153) and put the required number of hemostatic composition to the wound.

STERILITY. Another embodiment of the invention provides that a “device”, as one possible variants of hemostatic device—device (199), consisting of any of the above hemostatic devices or hemostatic compositions are placed in a sealed sterile package the shape of which corresponds to the form “of device”. Before applying any of the mentioned above “devices” or “compositions” package should be opened and get out of it “device” or “composition”.

The essence of the invention is also explained by specific examples producing of hemostatic “agent”.

Hemostatic composition of agents, wherein simultaneously are present water-retaining, binder dust suppression, inorganic and organic hemostatic agents, and characterized by composition of agents in the following ratio of components:

Generalized quantitative content in the hemostatic composition wherein simultaneously contains water-retaining hemostatic, binder dust suppression, inorganic, organic hemostatic, and characterized in that said are contained in the following ratio:

the composition of hemostatic wherein simultaneously contains water-retaining hemostatic, binder dust suppression, inorganic, organic hemostatic, and characterized in that said are contained in the following ratio

Generalized quantitative content of hemostatic agent composition which simultaneously contains water-retaining hemostatic agent, binder dust suppression, inorganic, organic hemostatic agents, and characterized in that said are contained in the following ratio:

### Example 1

Generalized quantitative content in the hemostatic composition wherein simultaneously contains water-retaining hemostatic, binder dust suppression, inorganic, organic hemostatic, and characterized in that said are contained in the following ratio:

water-retaining hemostatic agent—from 0.001% to 30%,

binder dust suppression hemostatic agent—from 0.001% to 50%,

inorganic hemostatic agent—from 0.001% to 50%,

organic hemostatic agent—from 0.001% to 50%,

which is a suspension or solution or foam or paste or gel or powder, and in which the total content of hemostatic is less than 100% or 100%.

### Example 2

Generalized quantitative content in the hemostatic composition wherein simultaneously contains water-retaining hemostatic, binder dust suppression, inorganic, organic hemostatic, and characterized in that said are contained in the following ratio:

water-retaining hemostatic agent—30%,

binder dust suppression hemostatic agent—35%,

inorganic hemostatic agent—34.999%,

organic hemostatic agent—0.001%,

which is a powder in which the total content of water-retaining hemostatic, dust suppressing binder hemostatic, inorganic hemostatic, organic hemostatic agent, is 100%.

### Example 3

Generalized quantitative content in the hemostatic composition wherein simultaneously contains water-retaining hemostatic, binder dust suppression, inorganic, organic hemostatic, and characterized in that said are contained in the following ratio:

water-retaining hemostatic agent—0.001%,

binder dust suppression hemostatic agent—50%,

inorganic hemostatic agent—24.999%,

organic hemostatic agent—25%,

which is a powder in which the total content of water-retaining hemostatic, dust suppressing binder hemostatic, inorganic hemostatic, organic hemostatic agent, is 100%.

### Example 4

Generalized quantitative content in the hemostatic composition wherein simultaneously contains water-retaining hemostatic, binder dust suppression, inorganic, organic hemostatic, and characterized in that said are contained in the following ratio:

water-retaining hemostatic agent—29.999%,

binder dust suppression hemostatic agent—0.001%,

inorganic hemostatic agent—34.999%,

organic hemostatic agent—28%,

which is a gel in which the total content of water-retaining hemostatic agent, dust suppressing binder hemostatic agent, inorganic hemostatic agent, organic hemostatic agents are 100%.

### Example 5

Generalized quantitative content in the hemostatic composition wherein simultaneously contains water-retaining hemostatic, binder dust suppression, inorganic, organic hemostatic, and characterized in that said are contained in the following ratio:

water-retaining hemostatic agent—10%,

binder dust suppression hemostatic agent—31%,

inorganic hemostatic agent—0.001%,

organic hemostatic agent—44.999%.

which is a suspension in which the total content of water-retaining hemostatic agent, dust suppressing binder hemostatic agent, inorganic hemostatic agent, organic hemostatic agents are 86% and the rest (14%) is water.

### Example 6

Generalized quantitative content in the hemostatic composition wherein simultaneously contains water-retaining hemostatic, binder dust suppression, inorganic, organic hemostatic, and characterized in that said are contained in the following ratio:

water-retaining hemostatic agent—21%,

binder dust suppression hemostatic agent—33%,

inorganic hemostatic agent—25%,

organic hemostatic agent—44.999%, and

which is a paste in which the total content of water-retaining hemostatic agent, dust suppressing binder hemostatic agent, inorganic hemostatic agent, organic hemostatic agent is 100%.

### Example 7

Generalized quantitative content in the hemostatic composition wherein simultaneously contains water-retaining hemostatic, binder dust suppression, inorganic, organic hemostatic, and characterized in that said are contained in the following ratio:

water-retaining hemostatic agent—4%,

binder dust suppression hemostatic agent—48%,

inorganic hemostatic agent—28%,

organic hemostatic agent—20%,

which is a foam in which the total content of water-retaining hemostatic agent, dust suppressing binder hemostatic agent, inorganic hemostatic agent, organic hemostatic agent is 100%.

### Example 8

Generalized quantitative content in the hemostatic composition wherein simultaneously contains water-retaining hemostatic, binder dust suppression, inorganic, organic hemostatic, and characterized in that said are contained in the following ratio:

water-retaining hemostatic agent—3.5%,

binder dust suppression hemostatic agent—32%,

inorganic hemostatic agent—21%,

organic hemostatic agent—22%,

which is a solution in which the total content of water-retaining hemostatic agent, dust suppressing binder hemostatic agent, inorganic hemostatic agent, organic hemostatic agent is 78.5% and the rest (11.5%) is water.

### Example 9

Generalized quantitative content in the hemostatic composition wherein simultaneously contains water-retaining hemostatic, binder dust suppression, inorganic, organic hemostatic, and characterized in that said are contained in the following ratio:

water-retaining hemostatic agent (oxidized cellulose)—2%,

binder dust suppression hemostatic agent (glycerol)—11%

inorganic hemostatic agent (calcium chloride)—21.49%,

organic hemostatic agent (collagen)—0.1%, and

which is a solution in which the total content of water-retaining hemostatic agent, dust suppressing binder hemostatic agent, inorganic hemostatic agent, organic hemostatic agent is 35% and the rest (65%) is water (65 kg).

### Example 10

Generalized quantitative content in the hemostatic composition wherein simultaneously contains water-retaining hemostatic, binder dust suppression, inorganic, organic hemostatic, and characterized in that said are contained in the following ratio:

water-retaining hemostatic agent—(sodium alginate)—0.1%,

binder dust suppression hemostatic agent (glycerol)—0.1%,

inorganic hemostatic agent (titanium oxide)—20%,

organic hemostatic agent (vitamin K)—0.001%, and

which is a gel in which the total content of water-retaining hemostatic agent, dust suppressing binder hemostatic agent, inorganic hemostatic agent, organic hemostatic agent is 20.201% and the rest (79.799%) is water.

### Example 11

Generalized quantitative content in the hemostatic composition wherein simultaneously contains water-retaining hemostatic, binder dust suppression, inorganic, organic hemostatic, and characterized in that said are contained in the following ratio:

water-retaining hemostatic agent—30%,

binder dust suppression hemostatic agent—11%,

inorganic hemostatic agent—50%,

organic hemostatic agent—9%,

which is a powder in which the total content of water-retaining hemostatic, dust suppressing binder hemostatic, inorganic hemostatic, organic hemostatic agent, is 100%.

### Example 12

Generalized quantitative content in the hemostatic composition wherein simultaneously contains water-retaining hemostatic, binder dust suppression, inorganic, organic hemostatic, and characterized in that said are contained in the following ratio:

water-retaining hemostatic agent—0.001%,

binder dust suppression hemostatic agent—20%,

inorganic hemostatic agent—10%,

organic hemostatic agent—15%,

which is a suspension in which the total content of water-retaining hemostatic agent, dust suppressing binder hemostatic agent, inorganic hemostatic agent, organic hemostatic agent is 45.001% and the rest (54.999%) is water.

### Example 13

The hemostatic composition, wherein simultaneously contains glycerol (10 g) as water-retaining hemostatic agent, chitosan (17 g) as the binder dust suppression hemostatic, the gallic acid (21 g) as an organic hemostatic, the bentonite (28 g) as an inorganic hemostatic, and which produced as a suspension using 24 g of water.

### Example 14

The hemostatic composition, wherein simultaneously contains carboxymethyl cellulose (20 g), as water-retaining hemostatic agent, the sodium alginate (0.001 g) as the binder dust suppression hemostatic, the gelatin (5 g) as an organic hemostatic, the calcium orthophosphate (21 g) as an inorganic hemostatic, and which produced as a suspension using 43.999 g of water.

### Example 15

The hemostatic composition, wherein simultaneously contains carboxymethyl cellulose (20 g), as water-retaining hemostatic agent, the sodium alginate (0.001 g) as the binder dust suppression hemostatic, gallic acid (21 g) as an organic hemostatic, the bentonite (28 g) as an inorganic hemostatic, and which produced as a suspension using 43.999 g of water.

### Example 16

The hemostatic composition, wherein simultaneously contains carboxymethyl chitosan (30 g), as water-retaining hemostatic agent, the calcium alginate (50 g) as the binder dust suppression hemostatic, quercetin (0.001 g) as an organic hemostatic, the kaolin (19999 g) as an inorganic hemostatic, and which produced as a powder.

### Example 17

The hemostatic composition, wherein simultaneously contains the glycerol (5 g) as water-retaining hemostatic agent, the chitosan (30.5 g) as the binder dust suppression hemostatic, gallic acid (20.5 g) as an organic hemostatic, the bentonite (21 g) as an inorganic hemostatic, and which produced as a paste using 23 g of water.

### Example 18

The hemostatic composition, wherein simultaneously contains the carboxymethyl cellulose (0.009 g) as water-retaining hemostatic agent, the sodium alginate (0.001 g) as the binder dust suppression hemostatic, the gelatin (11 g) as an organic hemostatic, the calcium orthophosphate (31 g) as an inorganic hemostatic, and which produced as a suspension using 57.09 g of water.

### Example 19

The hemostatic composition, wherein simultaneously contains the chitosan (5 kg) as water-retaining hemostatic agent, the glycerol (31 kg) as the binder dust suppression hemostatic, calcium glycerophosphate (0.009 kg) as an organic hemostatic agent, calcium alginate (21 kg) as an inorganic hemostatic, and which produced as a solution using 42.991 kg of water.

### Example 20

The hemostatic composition, wherein simultaneously contains the chitosan (28 g) as water-retaining hemostatic agent, carboxymethyl cellulose (50 g) as the binder dust suppression hemostatic, the quercetin (0.001 g) as an organic hemostatic agent, the kaolin (21.999 g) as an inorganic hemostatic, and which produced as a powder.

### Example 21

Quantitative and qualitative content in the hemostatic composition wherein simultaneously contains water-retaining hemostatic, binder dust suppression, inorganic, organic hemostatic, and characterized in that said are contained in the following ratio:

water-retaining hemostatic agent (polyacrylic acid)—3%,

binder dust suppression hemostatic agent (glycerol)—30%,

inorganic hemostatic agent (bentonite)—20%,

organic hemostatic agent (tannin)—20%, and

which is a suspension in which the total content of water-retaining hemostatic agent, dust suppressing binder hemostatic agent, inorganic hemostatic agent, organic hemostatic agent with following ratio of components, is 73% and the rest (27%) is water (37 g).

### Example 22

Quantitative and qualitative content in the hemostatic composition wherein simultaneously contains water-retaining hemostatic, binder dust suppression, inorganic, organic hemostatic, and characterized in that said are contained in the following ratio:

water-retaining hemostatic agent (oxidized cellulose)—2.1%,

binder dust suppression hemostatic agent (polyvinyl alcohol)—0.01%,

inorganic hemostatic agent (calcium phosphate)—19%,

organic hemostatic agent (gelatin)—0.09% and

which is a suspension in which the total content of water-retaining hemostatic agent, dust suppressing binder hemostatic agent, inorganic hemostatic agent, organic hemostatic agent with following ratio of components is 21.2% and the rest (78.8%) is water (78.8 g).

### Example 23

Quantitative and qualitative content in the hemostatic composition wherein simultaneously contains water-retaining hemostatic, binder dust suppression, inorganic, organic hemostatic, and characterized in that said are contained in the following ratio:

water-retaining hemostatic agent (sodium alginate)—0.02%,

binder dust suppression hemostatic agent (glycerol)—30%,

inorganic hemostatic agent (barium titanate)—17%,

organic hemostatic agent (polyvinylpyrrolidone)—20%, and

which is a suspension in which the total content of water-retaining hemostatic agent, dust suppressing binder hemostatic agent, inorganic hemostatic agent, organic hemostatic agent with following ratio of components is 67.02% and the rest (32.98%) is water (32.98 g).

### Example 24

Quantitative and qualitative content in the hemostatic composition wherein simultaneously contains water-retaining hemostatic, binder dust suppression, inorganic, organic hemostatic, and characterized in that said are contained in the following ratio:

water-retaining hemostatic agent (modified starch)—2.9%,

binder dust suppression hemostatic agent (glycerol)—29.01%

inorganic hemostatic agent (kaolin)—0.09%,

organic hemostatic agent (tannin)—19%, and

which is a suspension in which the total content of water-retaining hemostatic agent, dust suppressing binder hemostatic agent, inorganic hemostatic agent, organic hemostatic agent with following ratio of components is 51% and the rest (49%) is water (49 g).

### Example 25

Quantitative and qualitative content in the hemostatic composition wherein simultaneously contains water-retaining hemostatic, binder dust suppression, inorganic, organic hemostatic, and characterized in that said are contained in the following ratio:

water-retaining hemostatic agent (tara gum)—2.1%,

binder dust suppression hemostatic agent (polyethylene glycol)—15%,

inorganic hemostatic agent (calcium phosphate)—10%,

organic hemostatic agent (gallic acid)—11.9%, and

which is a suspension in which the total content of water-retaining hemostatic agent, dust suppressing binder hemostatic agent, inorganic hemostatic agent, organic hemostatic agent with following ratio of components is 39% and the rest (61%) is water (61 g).

### Example 26

Quantitative and qualitative content in the hemostatic composition wherein simultaneously contains water-retaining hemostatic, binder dust suppression, inorganic, organic hemostatic, and characterized in that said are contained in the following ratio:

water-retaining hemostatic agent (iron polyacrylate)—0.04%,

binder dust suppression hemostatic agent (pectin)—0.06%,

inorganic hemostatic agent (barium sulfate)—19%,

organic hemostatic agent (tannin)—18.9%, and

which is a suspension in which the total content of water-retaining hemostatic agent, dust suppressing binder hemostatic agent, inorganic hemostatic agent, organic hemostatic agent with following ratio of components is 28% and the rest (72%) is water (72 g).

### Example 27

Quantitative and qualitative content in the hemostatic composition wherein simultaneously contains water-retaining hemostatic, binder dust suppression, inorganic, organic hemostatic, and characterized in that said are contained in the following ratio:

water-retaining hemostatic agent (carboxymethyl cellulose)—2.5%,

binder dust suppression hemostatic agent (glycerol)—28.4%

inorganic hemostatic agent (silicon dioxide)—0.02%,

organic hemostatic agent (polyvinyl acetate)—0.08%, and

which is a suspension in which the total content of water-retaining hemostatic agent, dust suppressing binder hemostatic agent, inorganic hemostatic agent, organic hemostatic agent with following ratio of components is 31% and the rest (69%) is water (69 g).

### Example 28

Quantitative and qualitative content in the hemostatic composition wherein simultaneously contains water-retaining hemostatic, binder dust suppression, inorganic, organic hemostatic, and characterized in that said are contained in the following ratio:

water-retaining hemostatic agent (Arabian gum)—0.01%,

binder dust suppression hemostatic agent (glycerol)—15.49%

inorganic hemostatic agent (zeolite)—15%,

organic hemostatic agent (tannin)—18.5%, and

which is a suspension in which the total content of water-retaining hemostatic agent, dust suppressing binder hemostatic agent, inorganic hemostatic agent, organic hemostatic agent with following ratio of components is 49% and the rest (51%) is water (51 g).

### Example 29

Quantitative and qualitative content in the hemostatic composition wherein simultaneously contains water-retaining hemostatic, binder dust suppression, inorganic, organic hemostatic, and characterized in that said are contained in the following ratio:

water-retaining hemostatic agent (agar)—3%,

binder dust suppression hemostatic agent (polyvinyl alcohol)—12.59%,

inorganic hemostatic agent (calcium tripolyphosphate)—14.4%,

organic hemostatic agent (hyaluronic acid)—0.01%, and

which is a suspension in which the total content of water-retaining hemostatic agent, dust suppressing binder hemostatic agent, inorganic hemostatic agent, organic hemostatic agent with following ratio of components, is 30% and the rest (70%) is water (70 g).

### Example 30

Quantitative and qualitative content in the hemostatic composition wherein simultaneously contains water-retaining hemostatic, binder dust suppression, inorganic, organic hemostatic, and characterized in that said are contained in the following ratio:

water-retaining hemostatic agent (gelatin)—2%,

binder dust suppression hemostatic agent (glycerol)—20%,

inorganic hemostatic agent (aluminum oxide)—12%,

organic hemostatic agent (polyvinylpyrrolidone)—20%, and

which is a suspension in which the total content of water-retaining hemostatic agent, dust suppressing binder hemostatic agent, inorganic hemostatic agent, organic hemostatic agent with following ratio of components is 54% and the rest (46%) is water (46 g).

### Example 31

Quantitative and qualitative content in the hemostatic composition wherein simultaneously contains water-retaining hemostatic, binder dust suppression, inorganic, organic hemostatic, and characterized in that said are contained in the following ratio:

water-retaining hemostatic agent (methyl ester of polyacrylic acid)—1.2%,

binder dust suppression hemostatic agent (carboxymethyl cellulose)—0.1%,

inorganic hemostatic agent (calcium hydroxide)—11%,

organic hemostatic agent (chitosan)—0.7%, and

which is a suspension in which the total content of water-retaining hemostatic agent, dust suppressing binder hemostatic agent, inorganic hemostatic agent, organic hemostatic agent with following ratio of components is 13% and the rest (87%) is water (87 g).

### Example 32

Quantitative and qualitative content in the hemostatic composition wherein simultaneously contains water-retaining hemostatic, binder dust suppression, inorganic, organic hemostatic, and characterized in that said are contained in the following ratio:

water-retaining hemostatic agent (dextrin)—0.2%,

binder dust suppression hemostatic agent (glycerol)—20%

inorganic hemostatic agent (sodium silicate)—10.8%,

organic hemostatic agent (polyvinylpyrrolidone)—20%, and

which is a suspension in which the total content of water-retaining hemostatic agent, dust suppressing binder hemostatic agent, inorganic hemostatic agent, organic hemostatic agent with following ratio of components is 51% and the rest (49%) is water (49 g).

### Example 33

Quantitative and qualitative content in the hemostatic composition wherein simultaneously contains water-retaining hemostatic, binder dust suppression, inorganic, organic hemostatic, and characterized in that said are contained in the following ratio:

water-retaining hemostatic agent (polyvinyl acetate)—1.9%,

binder dust suppression hemostatic agent (glycerol)—19.1%,

inorganic hemostatic agent (aluminum oxide)—0.5%,

organic hemostatic agent (tannin)—18.5%, and

which is a suspension in which the total content of water-retaining hemostatic agent, dust suppressing binder hemostatic agent, inorganic hemostatic agent, organic hemostatic agent with following ratio of components is 30% and the rest (70%) is water (70 g).

### Example 34

Quantitative and qualitative content in the hemostatic composition wherein simultaneously contains water-retaining hemostatic, binder dust suppression, inorganic, organic hemostatic, and characterized in that said are contained in the following ratio:

water-retaining hemostatic agent (tara gum)—1.7%,

binder dust suppression hemostatic agent (glycerol)—20%

inorganic hemostatic agent (bentonite)—10%,

organic hemostatic agent 13.3%, and

which is a suspension in which the total content of water-retaining hemostatic agent,

dust suppressing binder hemostatic agent, inorganic hemostatic agent, organic hemostatic agent with following ratio of components is 45% and the rest (65%) is water (65 g).

### Example 35

Quantitative and qualitative content in the hemostatic composition wherein simultaneously contains water-retaining hemostatic, binder dust suppression, inorganic, organic hemostatic, and characterized in that said are contained in the following ratio:

water-retaining hemostatic agent (pectin)—0.4%,

binder dust suppression hemostatic agent (polyethylene glycol)—0.6%,

inorganic hemostatic agent (zeolite)—18%,

organic hemostatic agent (tannin)—17%, and

which is a suspension in which the total content of water-retaining hemostatic agent, dust suppressing binder hemostatic agent, inorganic hemostatic agent, organic hemostatic agent with following ratio of components is 36% and the rest (64%) is water (64 g).

### Example 36

Quantitative and qualitative content in the hemostatic composition wherein simultaneously contains water-retaining hemostatic, binder dust suppression, inorganic, organic hemostatic, and characterized in that said are contained in the following ratio:

water-retaining hemostatic agent (cyclodextrin)—1.6%,

binder dust suppression hemostatic agent (glycerol) —19%,

inorganic hemostatic agent (oxidized coal)—0.4%,

organic hemostatic agent (sodium polyacrylate)—1%, and

which is a suspension in which the total content of water-retaining hemostatic agent, dust suppressing binder hemostatic agent, inorganic hemostatic agent, organic hemostatic agent with following ratio of components is 22% and the rest (78%) is water (78 g).

### Example 37

Quantitative and qualitative content in the hemostatic composition wherein simultaneously contains water-retaining hemostatic, binder dust suppression, inorganic, organic hemostatic, and characterized in that said are contained in the following ratio:

water-retaining hemostatic agent (carrageenan)—0.1%,

binder dust suppression hemostatic agent (polyvinyl alcohol)—12%

inorganic hemostatic agent (zirconium oxide)—13%,

organic hemostatic agent (tannin)—16.9%, and

which is a suspension in which the total content of water-retaining hemostatic agent, dust suppressing binder hemostatic agent, inorganic hemostatic agent, organic hemostatic agent with following ratio of components, is 42% and the rest (58%) is water (58 g).

### Example 38

Quantitative and qualitative content in the hemostatic composition wherein simultaneously contains water-retaining hemostatic, binder dust suppression, inorganic, organic hemostatic, and characterized in that said are contained in the following ratio:

water-retaining hemostatic agent (calcium alginate)—2%,

binder dust suppression hemostatic agent (glycerol)—11%,

inorganic hemostatic agent (titanium oxide)—14.9%,

organic hemostatic agent (collagen)—0.1%, and

which is a solution in which the total content of water-retaining hemostatic agent, dust suppressing binder hemostatic agent, inorganic hemostatic agent, organic hemostatic agent with following ratio of components is 28% and the rest (72.9%) is water (72.9 g).

### Example 39

Quantitative and qualitative content in the hemostatic composition wherein simultaneously contains water-retaining hemostatic, binder dust suppression, inorganic, organic hemostatic, and characterized in that said are contained in the following ratio:

water-retaining hemostatic agent (polyethylene glycol) 1.1%,

binder dust suppression hemostatic agent (glycerol)—11%

inorganic hemostatic agent (calcium chloride)—14.9%, and

organic hemostatic agent (ascorbic acid)—0.1%, and

which is a solution in which the total content of water-retaining hemostatic agent, dust suppressing binder hemostatic agent, inorganic hemostatic agent, organic hemostatic agent with following ratio of components, is 28% and the rest (72%) is water (72 g).

### Example 40

Quantitative and qualitative content in the hemostatic composition wherein simultaneously contains water-retaining hemostatic, binder dust suppression, inorganic, organic hemostatic, and characterized in that said are contained in the following ratio:

water-retaining hemostatic agent (carboxymethyl cellulose) 2%,

binder dust suppression hemostatic agent (glycerol) —18.4%

inorganic hemostatic agent (silicon dioxide)—0.02%,

organic hemostatic agent (polyvinyl acetate)—1.58%, and

which is a foam in which the total content of water-retaining hemostatic agent, dust suppressing binder hemostatic agent, inorganic hemostatic agent, organic hemostatic agent with following ratio of components, is 22% and the rest (78%)—water (78 g).

### Example 41

Suspension

“Solution 1” was prepared by dissolving of 18.5 g tannin in 10 g of water and 19.1 g of glycerol by heating. Further, to the “Solution 1” were added 0.5 g of aluminum oxide and suspended with stirring using ultrasonic bath to form a “Solution 2”. Then 1.9 g of polyvinyl acetate pour into a reactor with 50 g of water and stirred by heating until completely dissolving of polyvinyl acetate to form the “Solution 3”. Next to the “Solution 3” was added “Solution 2” with stirring to form homogeneous suspension (“Solution 4”). Then substrate was impregnated to the “Solution 4” by immersing for complete impregnation of entire surface of substrate. Then the impregnated substrate (impregnated “substrate” or impregnated “container”) is removed from the “Solution 4” and wring out excess of “Solution 4” and dried for required moisture.

### Example 42

Suspension

“Solution 1” was prepared by dissolving of 5 g tannin in 30 g of water and 10 g of glycerol by heating. Further, to the “Solution 1” were added 1 g of zeolite and suspended with stirring using ultrasonic bath to form a “Solution 2”. Then 2 g polyvinylpyrrolidone pour into a reactor with 52 g of water and stirred by heating until completely dissolving of polyvinylpyrrolidone to form the “Solution 3”. Next to the “Solution 3” was added suspension zeolite and tannin with stirring to form homogeneous suspension (“Solution 4”). The suspension “Solution 4” is applied to the substrate by spraying until complete uniform impregnation of entire surface of substrate. Then the substrate is dried to the required humidity.

### Example 43

Suspension

“Solution 1” was prepared by dissolving of 10 g gallic in 30 g of water and 15 g of glycerol by heating. Further, to the “Solution 1” were added 10 g of bentonite and suspended with stirring using ultrasonic bath to form a “Solution 2”. Then 3 g of sodium alginate pour into a reactor with 32 g of water and stirred by heating until completely dissolving of sodium alginate to form the “Solution 3”. Next to the “Solution 3” was added “Solution 2” with stirring to form homogeneous suspension (“Solution 4”). Next the “Solution 4” was impregnated in the substrate using the slot die techniques (Slot-die coating process), rolled and dried to the required humidity.

### Example 44

Suspension

“Solution 1” was prepared by dissolving of 18.5 g tannin in 10 g of water and 19.1 g of glycerol by heating. Further, to the “Solution 1” were added 0.5 g of aluminum oxide and suspended with stirring using ultrasonic bath to form a “Solution 2”. Then 1.9 g of polyvinyl acetate pour into a reactor with 50 g of water and stirred by heating until completely dissolving of polyvinyl acetate to form the “Solution 3”. Next to the “Solution 3” was added “Solution 2” with stirring to form homogeneous suspension (“Solution 4”). The suspension (“Solution 4”) applied to the substrate by smearing (with a roller, brush, etc.) until complete uniform impregnation of entire surface of substrate. Then the substrate is dried to the required humidity.

### Example 45

Solution

“Solution 1” was prepared by dissolving of 18.5 g tannin in 10 g of water and 19.1 g of glycerol by heating. To the “Solution 1” were added 0.5 g of calcium chloride and dissolved with stirring using ultrasonic bath to form a “Solution 2”. Then 1.9 g of polyvinyl acetate pour into a reactor with 50 g of water and stirred by heating until completely dissolving of polyvinyl acetate to form the “Solution 3”. Next “Solution 3” was added to the “Solution 2” with stirring to form “Solution 4”. Then substrate was impregnated to the “Solution 4” by immersing for complete impregnation of entire surface of substrate. Then the impregnated substrate is removed from the solution and wring out excess of “Solution 4” and dried for required moisture.

### Example 46

Solution

“Solution 1” was prepared by dissolving of 5 g tannin in 30 g of water and 10 g of glycerol by heating. To the “Solution 1” were added 1 g of calcium chloride and dissolved with stirring using ultrasonic bath to form a “Solution 2”. Then 2 g polyvinylpyrrolidone pour into a reactor with 52 g of water and stirred by heating until completely dissolving of polyvinylpyrrolidone to form the “Solution 3”. Next “Solution 3” was added to the “Solution 2” with stirring to form “Solution 4”. The “Solution 4” is applied to the substrate by spraying until complete uniform impregnation of entire surface of substrate. Then the substrate is dried to the required humidity.

### Example 47

Solution

“Solution 1” was prepared by dissolving of 10 g gallic in 30 g of water and 15 g of glycerol by heating. Further, to the “Solution 1” were added 10 g of calcium glycerophosphate and suspended with stirring using ultrasonic bath to form a “Solution 2”. Then 3 g of sodium alginate pour into a reactor with 32 g of water and stirred by heating until completely dissolving of sodium alginate to form the “Solution 3”. Next to the “Solution 3” was added “Solution 2” with stirring to form homogeneous suspension (“Solution 4”). Next the “Solution 4” was impregnated in the substrate using the slot die techniques (Slot-die coating process), rolled and dried to the required humidity.

### Example 48

Gel Slot-Die

“Solution 1” was prepared by dissolving of 20 g tannin in 20 g of water and 4 g of glycerol by heating. To the “Solution 1” were added 10 g of titanium dioxide and suspended with stirring using ultrasonic bath to form dioxide titanium suspension a “Solution 2”. The substrate sample was immersed to the suspension and thoroughly soaked in the Solution for complete impregnation of entire surface of substrate.

Then the impregnated substrate is removed from the “Solution 2” and wring out excess of solution. Then 3 g of guar gum pour into a container reactor with 43 g of water and stirred by heating until completely dissolving of guar gum to form the “Solution 3”. Next the “Solution 3” is applied to the wet or dry substrate using the slot-die techniques (Slot-die coating process), rolled and dried to the required humidity.

### Example 49

Spray Foam

“Solution 1” was prepared by dissolving of 10 g of ellagic acid in 20 g of water and 5 g of glycerol by heating. Further, to the “Solution 1” were added 5 g of kaolin and suspended with stirring using ultrasonic bath to form a “Solution 2”. The substrate sample was immersed to the suspension (“Solution 2”) and thoroughly soaked for complete impregnation of entire surface of substrate. Then the impregnated substrate is removed from the “Solution 2” and wring out excess of solution. Then 1 g of chitosan pour into a container reactor with 54 g of water and 5 g of acetic acid (99.8%) and stirred by heating until completely chitosan dissolved to form the “Solution 3”. Next the “Solution 3” as a foam is applied to the wet or dry substrate by spraying, wring out excess of solution “Solution 3” and dried to the required moisture.

1. A hemostatic composition comprising a water-retaining hemostatic agent, a binder dust suppression hemostatic agent, an inorganic hemostatic agent, and an organic hemostatic agent, and having the following ratio of agents:
water-retaining hemostatic agent—from 0.001% to 30%,
binder dust suppression hemostatic agent—from 0.001% to 50%,
inorganic hemostatic agent—from 0.001% to 50%,
organic hemostatic agent—from 0.001% to 50%,
and in which the total content of hemostatic agents is less than or equal to 100%.

2. The hemostatic composition according to claim 1, in a form of solution or suspension or foam or gel or paste or powder.

3. The hemostatic composition, according to claim 1, wherein the water-retaining hemostatic agent contains a compound selected from the group consisting of but not limited to polysaccharides and its derivatives, in particular, carboxymethyl cellulose and/or its salts and/or polyols including glycerol, gums, in particular, locust bean, guar, xanthan, pectins; as binder dust suppression hemostatic agent contains a compound selected from the group consisting of but not limited to synthetic and/or natural, including modified polysaccharides, in particular, carboxymethyl cellulose, starch, agar-agar, gum arabic, dextrin, polyols in particular, glycerol, sorbitol, xylitol, maltol, polymer polyols, glycerol derivatives preferably, but not limited to propylene glycols, glyceryl triacetates and/or cyclic alcohols, preferably, but not limited to menthol, eugenol and combinations thereof; as organic hemostatic agent containing a compound selected from the group consisting of but not limited to ε-caproic acid, tranexamic acid, amben, fibrin, polyphenols and/or its components, in particular, tannin and/or tannic and/or gallic and/or digallic acid and/or flavonoids, in particular, rutin and/or quercetin, and/or preferably selected from the group of water-soluble polymers, including natural polymers, including chemically modified natural polymers, preferably selected from the group of cellulose derivatives, gelatin, gelatinized starch, polyvinylpyrrolidone, dextrose, pectin, chitosan, agar-agar, gum arabic, collagen, polyvinyl alcohol, polyacrylic acid, and its salts, silicone, polyvinyl acetate and/or group of polyols, preferably selected from the group glims, glycerol and its esters and/or plant extracts and/or combinations thereof; as inorganic hemostatic agent includes water-insoluble and/or sparingly soluble oxides of natural and/or synthetic origin, chosen preferably, but not limited to oxides of titanium, silicon, aluminum, etc., clays selected preferably, but not limited to attapulgite, kaolin, bentonite, etc. and/or its combinations and/or minerals selected preferably, but not limited to zeolites, including that might be part of clays, metal salts selected preferably, but not limited to calcium, barium sulfate, titanates, phosphates, glycerophosphate, etc.

4. The hemostatic composition according to claim 1, having the following ratio of the components:
water-retaining hemostatic agent—from 0.01% to 3%,
binder dust suppression hemostatic agent—from 0.01% to 30%,
inorganic hemostatic agent—from 0.01% to 20%,
organic hemostatic agent—from 0.01% to 20%.

5. The hemostatic composition according to claim 1, having the following ratio of the components:
water-retaining hemostatic agent—from 0.1% to 2%,
binder dust suppression hemostatic agent—from 0.1% to 20%,
inorganic hemostatic agent—from 1% to 12%,
organic hemostatic agent—from 0.1% to 20%.

6. The hemostatic composition according to claim 1, wherein at least one of inorganic hemostatic agents has radiographic properties.

7. The hemostatic composition according to claim 1, wherein the inorganic hemostatic agent that has radiographic properties, selected preferably, but not limited to barium sulfate, phosphate and titanate, including its hydrates.

8. The hemostatic composition of claim 1, wherein the packaging is sealed, which ensures sterility throughout its shelf life.

9. A hemostatic device comprising the composition of hemostatic agents according to claim 1 and a container that keeps hemostatic composition.

10. The hemostatic device according to claim 9, wherein the container is a product or device which is material to any selected from the following group: cotton, silk, wool, plastic, cellulose, rayon; polymer, e.g. nylon, polyethylene, polypropylene, polyester, polycarbonate, etc.; metal, glass, organic matter; a mixture of the above; woven, non-woven, knitted; film to water-permeable and/or water non-permeable.

11. The hemostatic device according to claim 9, wherein the container is a “two-dimensional”.

12. The hemostatic device according to claim 11, wherein the container has a regular and/or regular network structure and/or additional holes of any shape, preferably, but not limited to oval, square, rhombic.

13. The hemostatic device according to claim 11, wherein the container is a bandage, napkin, film that further can be folded and/or twisted and/or convoluted and/or stitched and/or glued.

14. The hemostatic device according to claim 9, wherein the container is a “three-dimensional”.

15. The hemostatic device according to claim 14, wherein the container is a vessel containing the composition of hemostatic agents or medications such as tampon, sponge etc.

16. The hemostatic device according to claim 9, wherein at least some part of container for the composition of hemostatic agents has a waterproof shell.

17. The hemostatic device according to claim 16, wherein at least some part of container for the composition of hemostatic agents has a waterproof shell that is filled with a composition of hemostatic agents in the form of a gel, foam, paste, suspension, solution, powder.

18. The hemostatic device according to claim 9, wherein at least a portion of the capacity for the composition of hemostatic agents presented by flexible carrier e.g. gauze is fibrous and/or mesh and/or a structure with air holes, incorporate composition of hemostatic agents in any sequence in any suitable way, particularly impregnated with a solution and/or suspension composition of hemostatic agents used spray solution and/or suspension composition of hemostatic agents, and/or use “slot-die” process for applying the solution and/or suspension composition of hemostatic agents and/or using smears carrier solution and/or suspension of the composition of hemostatic agents and/or using any combination of these methods, dried to the required moisture.

19. The hemostatic device according to claim 9, wherein it has an arbitrary way connected radiographic material presented preferably, but not limited to thread or strip or ribbon selected preferably, but not limited to polymers containing mainly barium compounds.

20. The hemostatic device according to claim 9, wherein additionally to composition of the said hemostatic agents applied pharmaceutically active composition preferably, but not limited to antibiotics, antifungal agents, antimicrobial agents, anti-inflammatory agents, analgesics, antihistamines, and oxides, hydroxides, salts, coordination compounds containing ions of copper, zinc, gold, silver, iron, calcium and/or nanoparticles of copper, gold, silver etc. and combinations thereof.

21. The hemostatic device according to claim 9, wherein the container is a vessel that contains composition of hemostatic agents in the form of a gel, foam, paste, suspension, solution, powder and/or hemostatic devices in form of napkin, bandage and/or its fragments thereof that have the specific geometry.

22. The hemostatic device according to claim 21, wherein the composition of hemostatic agents further comprises at least one foaming component.

23. The hemostatic device according to claim 21, wherein the container is presented by tube, balloon, syringe.

24. The hemostatic device according to claim 17, wherein the walls of the container produced of at least one layer of a flexible carrier in the form of gauze, which is fibrous and/or mesh and/or has a structure with holes, which is incorporated with the composition of hemostatic agents.

25. The hemostatic device according to claim 17, wherein the outer walls of the container produced of at least one layer of a flexible container in the form of gauze, which is fibrous and/or mesh and/or has a structure with holes and inner walls made of at least one layer of said container incorporated with the composition of hemostatic agents and wherein inner volume of container filled with the composition of hemostatic agents and/or fragments of “two-dimensional” hemostatic devices.

26. The hemostatic device according to claim 24, wherein it is connected to the flexible applicator in the form of a thread or strip to the other end of which is connected ring or washer for easy removal from the wound.

27. The hemostatic device “napkin” according to claim 12, wherein is a napkin of convenient form, preferably, but not limited to square or rectangular or oval that is pressed to the surface of the wound and/or placed inside the wound opening.

28. The hemostatic device “bandage” according to claim 12, wherein it is in the form of bandage tape corrugated repeatedly across the longitudinal axis of the original strip bandages.

29. The hemostatic device “bandage” according to claim 28, wherein the bandage repeatedly corrugated successively or alternately at an angle, preferably, but not limited to 45o, across the longitudinal axis of the original strip bandages serially and/or in opposite directions and combinations thereof.

30. The hemostatic device “bandage” according to claim 12, wherein the device is in the form of bandages tape rolled into a roll.

31. The hemostatic device according to claim 9, wherein the container is a waterproof tape with or without additional holes of any shape, preferably, but not limited to oval, square, rhombic and could be produced particularly of polyvinyl alcohol, gelatin, calcium alginate and incorporated with the composition of hemostatic agents.

32. The hemostatic device according to claim 9, wherein the container itself reveals additional hemostatic effect.

33. The hemostatic device “tampon” according to claim 12, wherein the said device is a “three-dimensional” geometrical figure, preferably, but not limited to cylindrical shape.

34. The hemostatic device “tampon” according to claim 33, wherein it has a cylindrical shape and obtained from the “two-dimensional” rectangular or square hemostatic device “napkin” type made by spiral twisting it diagonally to the diameter preferably, but not limited to about 5 mm, and which has a length preferably, but not limited to about 200-300 mm, made by cutting off unnecessary fragments from both ends and edges as formed preferably, but not limited to conical or oval shape, and attached by any suitable way, including gluing, stitching and others.

35. The hemostatic device “tampon” according to claim 14, wherein the specified device produced of at least one or two of the same and/or different fragments of hemostatic devices, including napkin, bandage, certain geometric shapes, interconnected by any suitable way, forming of this “three-dimensional” figure of special form.

36. The hemostatic device “tampon” according to claim 25, wherein the said device is made of a single piece of hemostatic devices, including napkin, bandage with a specific geometric shape in the form of tape and wherein the said strip material rolled into a roll, the free end of which is attached by any suitable way to the previous layer to prevent its unwinding and wherein the end of the roll formed by any suitable method for fixing the edge of strip and providing this conical end of the roll to facilitate its entering the wound opening and wherein the inner edge of the tape additionally fixed by any suitable method at one end of a flexible substrate in the form of rod diameter is preferably, but not limited to about 2 mm, and length preferably, but not limited to about 70 mm and wherein the total length of the said product preferably, but not limited to about 80 mm and wherein the twisted and fixed said “two-dimensional” device is preferably, but not limited to about 20 mm in length and preferably, but not limited to about 5 mm in diameter.

37. The hemostatic device “tampon” according to claim 25, wherein container is made of at least one layer of “two-dimensional” and/or at least one layer of “two-dimensional” hemostatic device and wherein two edges of said container and/or specified device cylindrically shaped in any suitable way, and where on the one side of cylinder edges connected in any suitable way and form bottom of conical or round shape, and wherein after filling the cylinder with “two-dimensional” hemostatic agent, chosen preferably, but not limited to napkin, bandage, including corrugated and/or fragments of these devices shaped in any suitable way into certain geometry, or composition of hemostatic agents in form of paste or dried suspension and combinations thereof and/or material selected from the group preferably, but not limited to cotton, viscose and other, and/or a mix of said device and/or said composition and/or materials, opening the bag closed in any suitable way; and wherein the cylinder is additionally compressed along and/or across the long axis of cylinder; and wherein the cylinder is preferably, but not limited to the following dimensions: about 40-70 mm in length and of about 7-20 mm in diameter, diameter of inner cavity is about 5-10 mm.

38. The hemostatic device “tampon” according to claim 37, wherein container is made of at least one layer of “two-dimensional” carrier and/or of at least one layer of “two-dimensional” hemostatic device is convoluted into cylindrical shape, repeating cylindrical shape of inner swab, formed at least of one fragment of “two-dimensional” hemostatic device and/or convoluted and/or twisted and/or compressed of several fragments of “two-dimensional” hemostatic device and fixed in any suitable way and connected with container in any suitable way preferably, but not limited to stitched to container along longitudinal axis of cylinder from one edge to other and wherein inner tampon additionally compressed and wherein free ends of layer of “two-dimensional” carrier and/or layer of “two-dimensional” hemostatic device are parallel to the longitudinal axis of cylinder freely sliding along the surface of the cylinder, that further providing the ability to several fold volume increase of compressed tampon after contact with blood in the wound, and wherein the tampon has preferably, but not limited to the following dimensions: about 40-70 mm in length and about 10-15 mm in diameter.

39. The hemostatic device “tampon” according to claim 37, wherein the said device further having applicator in the form of a thread or strip ensuring convenience of removal from the wound.

40. The hemostatic device “tampon” according to claim 37, wherein the tampon inside of introducer, which provides the convenience of plugging the wound.

41. The hemostatic device “tampon” according to claim 27, wherein the said device has a cylindrical shape and obtained from the “two-dimensional” hemostatic device “napkin” type with rectangular or square shape by twisting it along one edge a tight roll into diameter preferably, but not limited to about 5 mm and length preferably, but not limited to about 200-300 mm, which is formed by cutting off unnecessary fragments from both ends and edges of the cylinder formed preferably, but not limited to oval or conical shape and wherein the second edge of hemostatic device “napkin” attached along the long axis of the cylinder by any suitable way including gluing, stitching etc.

42. The hemostatic device “tampon” according to claim 27, wherein the container is made of at least one layer of “two-dimensional” hemostatic device,
and where the two edges of the said device are connected in cylindrical shape by any suitable way, and where on the one side of cylinder the edges connected by any suitable way and form cone bottom shape or round shape,
and wherein after filling the cylinder with “two-dimensional” hemostatic agent, chosen preferably, but not limited to napkin, bandage, including corrugated and/or fragments of these devices shaped in any suitable way into certain geometry, or composition of hemostatic agents in form of paste or dried suspension and combinations thereof and/or material selected from the group preferably, but not limited to cotton, viscose and other, and/or a mix of said device and/or said composition and/or materials, opening the bag closed in any suitable way; and wherein edges of the cylinder has conical or round shape; and wherein the cylinder is preferably, but not limited to the following dimensions: about 50-200 mm in length and about 7-20 mm in diameter, diameter of inner cavity is about 5-10 mm.

43. The hemostatic device “tampon pocket” according to claim 35, wherein it is made mainly in the form of a pocket, which is a container preferably of square or rectangular shape, made by any suitable manner of at least one layer, preferably, but not limited to 4-8 layers of “two-dimensional” hemostatic device bandage or napkin type, and where two walls of container connected on three sides (attached) in any suitable way, and the fourth side has a free hole in the middle has a cavity.

44. The hemostatic device “tampon pocket” according to claim 43, wherein through free hole located on the fourth side inner cavity of the said device any suitable way filled with “two-dimensional” hemostatic agent, chosen preferably, but not limited to napkin, bandage including corrugated and/or fragments of these said devices by any suitable way provided specific geometric shape and/or composition of hemostatic agents in the form of paste or dried slurry and combinations thereof, and/or mixtures of the said devices and hemostatic composition agents and/or its components.

45. The hemostatic device “tampon pocket” according to claim 44, wherein the “two-dimensional” hemostatic device is corrugated bandage with one end attached inside the pocket to its bottom, and the other looks outside, enabling the use of the said bandage as additional hemostatic device.

46. The hemostatic device “tampon pocket” according to claim 45, wherein in addition to the outer surface of one of the edges of the pocket hole attached by any suitable way to one end of elastic bandage or conventional bandage preferably, but not limited to about 5 meters in length, ensuring fixation of hemostatic agent to wounds and/or the imposition of compressive bandages.

47. The hemostatic device “tampon pocket” according to claim 46, wherein in addition to the free edge of elastic bandage or conventional bandage attached at least one special fastener to ensure reliable fixation on the wound.

48. The hemostatic device “tampon sac” according to claim 35, wherein it made in the form of mostly sac, is container preferably cylindrical form, made by suitable way of at least one layer of “two-dimensional” hemostatic device bandage or napkin, and where the bottom of the bag is preferably but not limited to conical or oval form, and the fourth side of it has a free hole in the middle of a cavity.

49. The hemostatic device “tampon sac” according to claim 48, wherein the sac corrugated along its longitudinal axis, ensuring an increase its length and volume upon filling with composition of hemostatic agents and/or hemostatic devices and/or their fragments and therefore better filling the volume and reliability plugging the wound.

50. The hemostatic device “tampon pad” according to claim 43, wherein, unattached to it hole from fourth side of pocket is closed (attached) by any suitable way.

51. The hemostatic device “tampon pad” according to claim 50, wherein to the said hemostatic device on the outer surface of one of the edges or in one of the joints connecting walls of the device additionally attached in any suitable way one end of an elastic bandage or conventional bandage with length preferably, but not limited to about 5 meters, ensuring fixation of hemostatic device to the wound and/or the imposition of compressive bandage.

52. The hemostatic device “tampon pad” according to claim 51, wherein to the free end of an elastic bandage or conventional bandage in any suitable way fixed at least one fastener that provides a secure fixation of hemostatic device to the wound.

53. The hemostatic device “tampon disc” according to claim 33, wherein, consisting of preferably, but not limited to 4-8 layers of round shape fragments of “two-dimensional” hemostatic agent, which stitched together in any way around the perimeter and form a geometrical figure disc.

54. The hemostatic device “tampon disc” according to claim 53, wherein further comprising at least one layer of moisture-absorbing material.

55. The hemostatic device “tampon ruff” according to claim 35, wherein, said device consists of a thin flexible rod preferably, but not limited to 2 mm in diameter and preferably, but not limited to 200 mm in length, and the end is tightly strung range hemostatic material with diameter preferably, but not limited to about 5 to 15 mm, and which form cylindrical column near 30 to 100 mm in length is fixed on both sides of the said rods stoppers and wherein the end of the device is pressed to the opening of the wound channel and gradually introduced into the latter.

56. The hemostatic device “window-type” bandage according to claim 28, wherein hemostatic agent is produced in the form of strip; and wherein hemostatic device consisting of strips of corrugated bandage with both ends connected in any suitable way to one non-elastic strip preferably, but not limited to rectangular shape; and wherein two edges of said strips are attached to each of the two strips of elastic bandage at a distance from each other preferably, but not limited to about 200 mm; and wherein two strips of elastic bandage placed along the edges of longitude axis of ribbon of said device; and wherein due to perpendicular arrangement of non-elastic bands and strips of elastic bandage formed fenestrated structure of said device; and wherein in each “window-type” placed a piece of corrugated bandage; and wherein said transverse strips are made of “two-dimensional” material preferably, but not limited to textiles; and wherein said hemostatic agent is an elastic bandage, consisting of multiple windows, to stop bleeding, which has properties of bandage directly compressing the wound.

57. The hemostatic combined device “window-type” bandage according to claim 56, wherein to the one end of the device namely non-elastic strip is attached elastic bandage or non-elastic bandage and/or at least one special lock preferably, but not limited to a special fastener.

58. The hemostatic combined device “window-type” bandage according to claim 50, wherein to the one of the edges of the device and is free non-elastic strips is attached special tampon, namely, the pad.

59. The hemostatic combined device “window-type” bandage according to claim 44, wherein to the one of the edges of the device, namely non-elastic strip is attached special tampon, namely, the pocket.

60. The hemostatic combined device “window-type” bandage according to claim 44, wherein to the one of the edges of the device namely non-elastic strip is attached special tampon, namely, the pocket with corrugated bandage inside, one edge of which peeps out of the hole of pocket.

61. The hemostatic device “tampon” according to claim 33, wherein, consists of equal size balls of hemostatic device, preferably, but not limited to diameter of 5 to 20 mm of strongly connected with strand or strip in distance of preferably, but not limited to about 5 mm to 50 mm is pressed to the surface of the wound is pressed to the surface of the wound the gradual introduction of interconnected balls one by one and is designed for plugging deep wound surfaces preferably, but not limited to narrow inlet.

62. The hemostatic device “tampon” according to claim 61, wherein one of its end strands or ribbons fixed to the washer serves as an applicator for easy removal of the said device from the wound.

63. The hemostatic device “tampon” according to claim 61, wherein device further comprises syringe type introducing device, consisting of a body in the form of a tube with a beveled edge; and wherein beveled edge at sharp angle directing applicator to the wound opening and application of hemostatic device to the channel and protected with cap; and wherein to the other end of the tube inserted rod with a piston, with firmly attached rubber washer of said hemostatic product; and wherein balls of said hemostatic device are tightly compressed in the cylinder of the syringe to the thickness preferably, but not limited to about 5 to 7 mm; and wherein upon pressing to piston and extrusion content by piston into wound hole this washer is on the wound edge and upon reverse movement of piston it disconnects from the piston and stay above the wound channel.

64. The hemostatic device “tampon” according to claim 61, wherein the hemostatic agent in the form of pellets derived from the “two-dimensional” hemostatic device any suitable way or the “two-dimensional” capacitance formed in the shape of balls every way possible, followed with ‘union fragments said device in the form of balls one by one together using threads and/or tapes in any suitable way.

65. The hemostatic device combined “tampon” according to claim 48, wherein further comprising introducer, mainly in the form of syringe filled with a solution, gel or suspension of hemostatic agents;
and where on the one hand syringe, such as opening of its conical edge corked with stopper that holds solution, suspension or gel of hemostatic composition inside the syringe before plunger is press on, but can be easily removed by pressing the plunger;
and where the piston has similar to a cone shape corresponding shape of the inner surface of the syringe and its end of conical shape, and provides quantitative squeezing of suspension or gel of hemostatic composition through the opening of syringe;
where the outer surface of the syringe installed ring freely moving (sliding) on the surface of the syringe and remains out of the wound;
and wherein on rubber ring on the specified registered one edge of the cylinder made from a fragment of “two-dimensional” hemostatic device with rectangular or square shape, two edges of which along the axis of the cylinder are interconnected, and where the cylinder tightly drawn accordion along the syringe body and thus where the second end of the cylinder is preferably conical or oval, which provides fixation of the edge of the cylinder around its perimeter at one point;
and wherein ring attached to the other end of the cylindrical bandage and easily glides with a bandage on the cylinder of the syringe by pressing the plunger of the syringe moves to the edge and upon glides of the cylinder it should firmly stopple (closing as diaphragm) to form directly in the wound closed from all sides bag with located inside it solution, suspension or gel of hemostatic composition absorbing moisture and increases in volume;
and where to avoid abuse corrugated shape bag has an additional protective cap that is removed immediately before application to the wound.

66. The hemostatic device combined “tampon” according to claim 65, wherein protective cap preventing disruption of corrugated shape of sac, made in the form of a cylinder, which bottom has at least four petals apart under pressure of the sac, which shifted from the introducer case to the extent of filling with solution, suspension or gel of hemostatic composition upon pressure on the piston rod, which provides sterile plugging the wound and where to avoid deformation of cap petals impaled on him an additional protective cap to be removed immediately before application to the wound.

67. The hemostatic device combined “tampon” according to claim 65, characterized in that: a cylinder filled with “two-dimensional” hemostatic device, chosen preferably, but not limited to napkin, bandage, including corrugated and/or fragments of these devices that any suitable way provided certain geometry or with hemostatic composition in the form of paste or dried slurry and combinations thereof, and/or a mixture of the above said devices and composition,
and where said devices and/or fragments thereof additionally compressed; and
where the edge of the cylinder through which the tampon is squeezed out of the cylinder, has cylindrical shape, and
wherein the edge of the cylinder is closed with round stopper,
and where the piston has a shape that corresponds shape of the inner surface of the syringe and the end of his round, and provides quantitative tampon squeezing through the opening of a syringe.

68. The hemostatic device combined “tampon” according to claim 67, wherein for the convenience of plugging the edge of the cylinder through which the tampon is squeezed out of the cylinder is beveled and has an ellipsoidal shape, and wherein the edge of the cylinder is closed by the stopper of ellipsoid shape.

69. The hemostatic device “patch” according to claim 27, wherein it composed of the tampon, which in any suitable way connected to preferably, but not limited to stretching or elastic polymer and/or plastic and/or woven and/or non-woven base preferably rectangular, square or oval form and wherein part of surface of said elastic base at least of one side covered with adhesive layer of pressure sensitive adhesive agent, securing its adhesion to patient's skin and wherein said device localized on patient's wound such as hemostatic device contacting with wound's surface and adhesive surface of elastic base fixing on the skin around wound and wherein said base is waterproof or perforated with pores securing evaporation of moisture from skin surface.

70. The hemostatic device “patch” according to claim 27, wherein the tampon is “two-dimensional” hemostatic device preferably napkin or fragment thereof having any suitable form preferably, but not limited to square, circle, rhomb and so on.

71. The hemostatic device “patch” according to claim 33, wherein the tampon is “tree-dimensional” hemostatic device preferably, but not limited to cylinder, ellipse, cube, disc and so on shape.

72. The hemostatic device “patch” according to claim 50, wherein the tampon is a “pad”.

73. The hemostatic device “patch” according to claim 43, wherein the tampon is “pocket” or “pocket” filled with z-gauze.

74. The hemostatic device “patch” according to claim 27, wherein at least a portion of at least one side of surface of container for hemostatic composition is applied with adhesive composition.

75. The hemostatic device according to claim 9, wherein the packaging is sealed, which ensures its sterility during shelf life.
